(19) United States
(12) Patent Application Publication
(10) Pub. No.: US 2007/0173454 A1
(43) Pub. Date: Jul. 26, 2007

(54) Methods for slowing senescence and treating and preventiing diseases associated with senescence

(75) Inventor: Richard L. Bowen, Raleigh, NC (US)

Correspondence Address: COVINGTON & BURLING, LLP;ATTN: PATENT DOCKETING, 1201 PENNSYLVANIA AVENUE, N.W., WASHINGTON, DC, 20004-2401, US

(73) Assignee: Voyager Pharmaceutical Corporation, Raleigh, NC (US)

(21) Appl. No.: 11/709,837

(22) Filed: Feb. 23, 2007

Related U.S. Application Data
(63) Continuation of application No. 10/321,579, filed on Dec. 18, 2002, now abandoned.
(60) Provisional application No. 60/340,502, filed on Dec. 19, 2001.
(60) Provisional application No. 60/385,577, filed on Jun. 5, 2002.
(60) Provisional application No. 60/369,857, filed on Apr. 5, 2002.
(60) Provisional application No. 60/385,576, filed on Jun. 5, 2002.
(60) Provisional application No. 60/383,624, filed on May 29, 2002.
(60) Provisional application No. 60/385,560, filed on Jun. 5, 2002.
(60) Provisional application No. 60/385,559, filed on Jun. 5, 2002.
(60) Provisional application No. 60/385,561, filed on Jun. 5, 2002.
(60) Provisional application No. 60/385,575, filed on Jun. 5, 2002.

(51) Int. Cl. A61K 38/23; A61K 38/09

(52) U.S. Cl. 514/12; 514/15

(57) ABSTRACT

The present invention relates to a method for slowing, preventing or delaying senescence or treating or preventing a disease associated with senescence by administering a therapeutically effective amount of at least one physiological agent that decreases or regulates the blood level, production, function or activity of LH or FSH, or that decreases or regulates the production or activity of activin, or that increases or regulates the blood level, production, function, or activity of inhibin or follistatin.

### RELATED APPLICATIONS

This application claims priority under 35 U.S.C. §119(e) to: U.S. Provisional Application No. 60/340,502, filed Dec. 19, 2001; U.S. Provisional Application No. 60/369,857, filed Apr. 5, 2002; U.S. Provisional Application No. 60/383,624, filed May 29, 2002; U.S. Provisional Application No. 60/385,577, filed Jun. 5, 2002; U.S. Provisional Application No. 60/385,576, filed Jun. 5, 2002; U.S. Provisional Application No. 60/385,560, filed Jun. 5, 2002; U.S. Provisional Application No. 60/385,559, filed Jun. 5, 2002; U.S. Provisional Application No. 60/385,561, filed Jun. 5, 2002; and U.S. Provisional Application No. 60/385,575, filed Jun. 5, 2002, the entirety of each of which is incorporated herein by reference.

### BACKGROUND OF THE INVENTION

### Field of the Invention

The present invention relates to a method for slowing, preventing or delaying senescence or treating or preventing a disease associated with senescence. More particularly, the present invention relates to a method for slowing, preventing or delaying senescence, or treating or preventing a disease associated with senescence, by administering a therapeutically effective amount of at least one physiological agent that decreases or regulates the blood level, production, function or activity of gonadotropins—leutinizing hormone (“LH”) or follicle stimulating hormone (“FSH”)—or that decreases or regulates the blood level, production, function or activity of activin, or that increases or regulates the blood level, production, function, or activity of inhibin or follistatin.

### SUMMARY OF THE INVENTION

The present invention encompasses a method of slowing, preventing or delaying senescence, or treating or preventing a disease associated with senescence, or inhibiting or preventing upregulation of the cell cycle, or decreasing the mitogenic index, or inhibiting the shortening of telomeres, in a subject, by administering an agent that decreases or regulates the blood level, production, function, or activity of LH or FSH (an “LH/FSH-inhibiting agent”).

The present invention further encompasses a method of slowing, preventing or delaying senescence, or treating or preventing a disease associated with senescence, or inhibiting or preventing upregulation of the cell cycle, or decreasing the mitogenic index, or inhibiting the shortening of telomeres, in a subject, by administering an agent that decreases or regulates the blood level, production, function or activity of activin (an “activin-inhibiting agent”).

In addition, the present invention encompasses a method of slowing, preventing or delaying senescence, or treating or preventing a disease associated with senescence, or inhibiting or preventing upregulation of the cell cycle, or decreasing the mitogenic index, or inhibiting the shortening of telomeres, in a subject, by administering an agent that increases or regulates the blood level, production, function, or activity of follistatin (a “follistatin-promoting agent”).

The present invention further encompasses a method of slowing, preventing or delaying senescence, or treating or preventing a disease associated with senescence, or inhibiting or preventing upregulation of the cell cycle, or decreasing the mitogenic index, or inhibiting the shortening of telomeres, in a subject, by administering an agent that increases or regulates the blood level, production, function, or activity of inhibin (an “inhibin-promoting agent”).

The present invention further encompasses a method of slowing, preventing or delaying senescence, or treating or preventing atherosclerosis, osteoporosis, or brain damage associated with acute brain injury, by administering an agent that prevents or inhibits cells from entering into the cell cycle (“cell cycle inhibitors”). Such agents include, but are not limited to, for example, low density lipoprotein receptor related protein receptor associated protein (“RAP”); a vaccine or antibody against proteins involved in promoting cell division (e.g. against cell cycle proteins such as CDK); taxol; vitamin A; hydroxyurea; colchicines; cholesterol lowering drugs, such as lovastatin or provastatin; and analogs, metabolites, precursors, and salts of these agents.

The present invention further encompasses a method of determining a mitogenic index in a subject, comprising: providing a test sample comprising a first plurality of cells from a standardized cell line in a standard growth medium; collecting a tissue sample from a subject; adding the tissue sample to the test sample to form a combined sample; measuring cell proliferation of the combined sample; providing a control sample comprising a second plurality of cells from the standardized cell line in the standard growth medium; measuring cell proliferation of the control sample; and comparing the cell proliferation of the control sample and the cell proliferation of the combined sample.

The present invention also encompasses a system for measuring a mitogenic index in a subject, comprising: a test sample comprising a first plurality of cells from a standardized cell line in a standard growth medium; means for collecting a tissue sample from a subject; means for adding the tissue sample to the test sample to form a combined sample; means for measuring cell proliferation of the combined sample; a control sample comprising a second plurality of cells from the standardized cell line in the standard growth medium; means for measuring cell proliferation of the control sample; and means for comparing the cell proliferation of the control sample and the cell proliferation of the combined sample.

### BRIEF DESCRIPTION OF THE DRAWINGS

FIG. 1 is a schematic diagram showing the pattern of gonadotropin secretion during the course of a normal, healthy person from conception until death.

FIG. 2 illustrates the effect of various amounts of LH on the proliferation of BrdU labeled neuroblastoma cells.

FIG. 3 illustrates and compares the proliferation of neuroblastoma cells exposed to leuprolide to a control sample not exposed to leuprolide.

FIG. 4 illustrates the blood level of follistatin for a constant rate infusion of 10 mcg/kg/hour over a 10 hour period and over a 24 hour period.

### DETAILED DESCRIPTION

In this specification, by “senescence” is meant any change in the function of an organism, or any of its tissues, that occurs concomitantly with a decline in reproductive function after the period of greatest reproductive function, which in humans typically corresponds to about 18 to 35 years of age. By “disease associated with senescence” is meant any disease, disorder, degeneration, tissue loss, or other unhealthy or abnormal condition caused by, linked to, or otherwise associated with senescence. Examples of diseases associated with senescence include, but are not limited to, artherosclerosis, brain cancer (including but are not limited to neuroma, anaplastic astrocytoma, neuroblastoma, glioma, glioblastoma multiforme, astrocytoma, meningioma, pituitary adenoma, primary CNS lymphoma, medulloblastoma, ependymoma, sarcoma, oligodendroglioma, medulloblastoma, spinal cord tumor, and schwannoma), polyps of the colon and colorectal cancer, myeloproliferative diseases (including but not limited to Hodgkin's disease, multiple myeloma, lymphoma, transient myeloproliferative disorder (TMD) (also known as transient myeloproliferative syndrome), congenital transient leukemia, congenital leukemoid reaction, transient leukaemoid proliferation, transient abnormal myelopoiesis, acute myeloid leukemia (AML), acute megakaryoblastic leukemia (AMKL) (also known as erythro-megakaryoblastic leukaemia); common B-lineage acute lymphoblastic leukemia (ALL), polycythemia, thrombocythemia, myelodysplastic syndromes, myelofibrosis, hypereosinophilic syndrome (HES), chronic lymphocytic leukemia, prolymphocytic leukemia, hairy-cell leukemia, chronic myelogenous leukemia, other leukemias, and other myelogenous cancers), osteoarthritis, osteoporosis, neoplasms, cataracts, macular degeneration, hearing loss, stroke, periodontal disease, osteopenia, peripheral neuropathy, COPD, hypertension, type 2 diabetes, sarcopenia, hypertension, primary pulmonary hypertension, congestive heart failure, left ventricular hypertrophy, cardiac valvular disease, esophagitis, esophageal stricture, gastroparesis, chronic pancreatitis, hypercholesterolemia, hypertriglyceridemia, cirrhosis of the liver, hepatitis, cholelithiasis, cholecystitis, ulcerative colitis, inflammatory bowel disease, Crohn's disease, fibromyalgia, obesity, renal failure, proteinuria, gout, hyperuricemia, membranous nephropathy, polyarteritis nodosa, polymyalgia rheumatica, rheumatoid arthritis, progressive systemic sclerosis, spinal stenosis, spinal cord injury, migraine headaches, male pattern baldness, sarcoidosis, Wegener granulomatosis, amyloidosis, dermatomyositis, graft versus host disease, systemic lupus erythematosus, seborrheic dermatitis, psoriasiform eczematous dermatitis, papulosquamous eczematous dermatitis, psoriasis, seborrheic keratosis, anagen effluvium, dysphagia, Barrett esophagus, achalasia, Chagas disease, facial neuropathy, trigeminal neuralgia, carpal tunnel syndrome, mitochondrial myopathies and encephalopathies, myasthenia gravis, traumatic brain injury, astrocytomas, oligodendrogliomas, meningiomas, schwannomas, pituitary adenomas, pineocytoma and pineoblastoma, primary central nervous system lymphoma, medulloblastomas, spinal cord tumors, paraneoplastic syndromes, anoxic encephalopathies, multiple sclerosis, transverse myelitis, Parkinson's disease, squamous cell carcinoma of the lung, adenocarcinoma of the lung, large cell carcinoma of the lung, small cell carcinoma of the lung, esophageal cancer, gastric cancer, pancreatic cancer, hepatocellular cancer, gallbladder carcinomas, colorectal cancer, Hodgkin's disease, non-Hodgkin's lymphoma, follicular lymphoma, small lymphocytic lymphoma, mantle cell lymphoma, marginal zone lymphoma, diffuse large cell lymphoma, Burkitt's and Burkitt's-like lymphoma, lymphoblastic lymphoma, peripheral T-cell lymphoma, large cell (T-cell and null) anaplastic lymphoma, primary anaplastic lymphoma, multiple myeloma, Ewing's sarcoma, chondrosarcomas, osteosarcomas, renal cell carcinoma, bladder carcinoma, testicular carcinoma, seminoma, nonseminoma, squamous cell carcinoma of the head and neck, salivary gland tumors, pneumoconioses, asbestosis, silicosis, coal worker's pneumoconiosis, berylliosis, malignant diffuse infiltrative lung disease, disease caused by pulmonary lymphangitic carcinomatosis, disease caused by alveolar cell carcinoma, chronic diffuse infiltrative lung disease of unknown etiology, sarcoidosis, idibphatic pulmonary fibrosis, desquamative interstitial pneumonia/respiratory bronchiolitis, interstitial lung disease, acute interstitial pneumonia, lymphocytic interstitial pneumonia, nonspecific interstitial pneumonia/fibrosis, bronchiolitis obliterans, Sjögren syndrome, mixed connective tissue disease, eosinophilic granuloma of the lung, allergic granulomatosis and angiitis, hypereosinophilic syndrome, osteoarthritis, spinal arthritis, ankylosing spondylitis, reactive arthritis (formerly known as Reiter syndrome), psoriatic arthritis, enteropathic arthritis, juvenile spondyloarthropathy, acne-associated arthritis, SAPHO (synovitis, acne, pustulosis, hyperostosis, osteitis) syndrome, Whipple disease, Paget's disease of bone, osteomalaci, decreased muscle mass, decreased skin elasticity, thinning of skin, decreased scalp hair growth, loss of subcutaneous collagen, decreased immune function, decreased lung function, loss of arterial elasticity, urinary incontinence, loss of renal function, brain damage associated with acute brain injury and reduced ejaculatory distance.

By “upregulation of the cell cycle” is meant an increased frequency or rate of cells entering into the cell cycle. By “cell cycle” is meant the process by which cells undergo chromosome replication and division to create new daughter cells. By “increased mitogenic stimulus” is meant an increase in the blood level, production, function or activity of a mitogenic promoting factor or a decrease in the blood level, production, function, or activity of mitogenic inhibiting factor. By “mitogenic promoting factor” is meant a compound that acts as an impetus for cells to enter into the cell cycle, including, but not limited to, LH, FSH, and activin. By “mitogenic inhibiting factor” is meant a compound that inhibits cells from entering into the cell cycle, either directly or by inhibiting the activity of a mitogenic stimulus, including, but not limited to, inhibin and follistatin. Throughout this application, the terms “upregulation of the cell cycle” and “increased mitogenic stimulus” are used interchangeably.

Mechanisms of Senescence and Diseases Associated with Senescence

The current prevailing theory of senescence is that as organisms and their tissues age, the rate of cells entering into the cell cycle continually declines, and that when a cell is no longer able to enter the cell cycle it becomes dysfunctional or dies. (Mathon N F, Lloyd A C, Cell senescence and cancer, Nature Rev Cancer December; 1(3):203-13 (2001)). In accordance with the present invention, and contrary to conventional teachings, senescence is caused by an upregulation of the cell cycle and/or increased mitogenic stimulus associated with a decline in reproductive function. For example, research has shown that the intestines of senescent rats have an increased rate of cell division. (E.g., Holt P R, Yeh K Y, Kotler D P, Altered controls of proliferation in proximal small intestine of the senescent rat, Proc Natl Sci USA April; 85(8):2771-5 (1988)). Similar findings have also been demonstrated in humans. (Ciccocioppo R, Di Sabatino A, Luinetti O, Rossi M, Cifone M G, Corazza G R. Descner E E, Small bowel enterocyte apoptosis and proliferation are increased in the elderly, Gerontology 2002 July-August; 48(4):204-8). Accordingly, it is an object of the present invention to slow senescence, or to prevent or delay the onset of senescence, by administering one or more agents that inhibit upregulation of the cell cycle.

In addition, in accordance with the present invention, and contrary to conventional teachings, diseases associated with senescence are caused by an upregulation of the cell cycle. An upregulation of the cell cycle may have different effects on different types of cells, leading to different diseases. For example, in some diseases associated with senescence, such as many cancers, cells have undergone mutations allowing them to divide and proliferate indefinitely. One mechanism by which these mutations occur is by an error in DNA transcription. Since DNA transcription occurs with every cell cycle, the more frequently cells cycle, the greater the probability of an error in DNA transcription, which could cause a mutation that transforms a healthy cell into a cancer cell. Therefore, not only does upregulation of the cell cycle increase the likelihood of a mutation occurring, but once a mutation has occurred, upregulation of the cell cycle contributes to cancer cells proliferating at an increased rate.

Some other diseases associated with senescence, such as those associated with many neuronal cells, arise from upregulation of the cell cycle of terminally differentiated cells (i.e., cells that are unable to complete the cell cycle). Upregulation of the cell cycle causes terminally differentiated cells to enter the cell cycle, but when these cells are unable to complete the cell cycle, they die or become dysfunctional, leading to a disease state. In yet some other diseases associated with senescence, such as atherosclerosis or osteoporosis, upregulation of the cell cycle causes otherwise healthy cells to proliferate at a rate greater than normal, leading to pathological consequences. Accordingly, it is an object of the present invention to treat or prevent diseases associated with senescence by administering an agent that inhibits an upregulation of the cell cycle.

According to the present invention, an increase in the blood level, production, function or activity of LH or FSH, or an increase in the blood level, production, function or activity of activin, or a decrease in the blood level, production, function or activity of inhibin or follistatin, contributes to an upregulation of the cell cycle related to senescence and/or diseases associated with senescence.

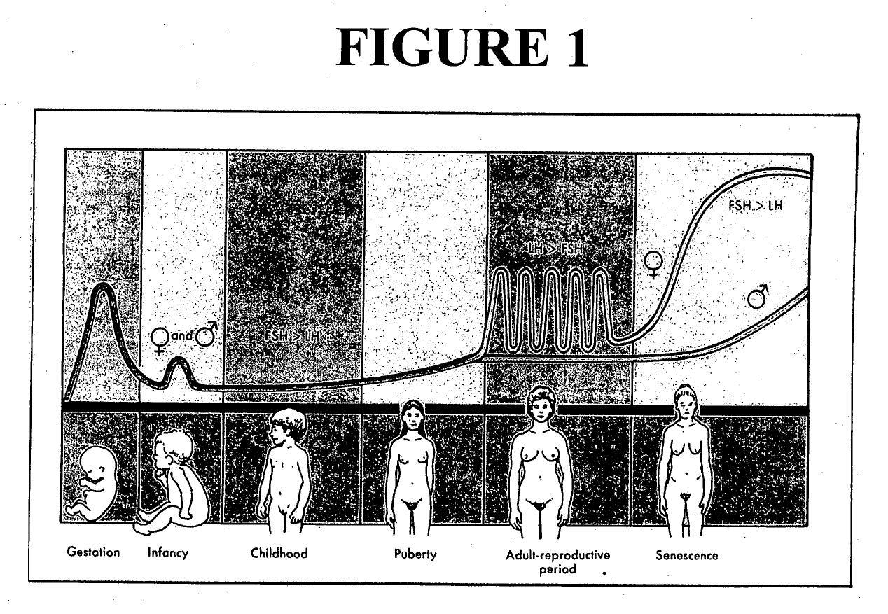

FIG. 1 is a schematic diagram showing the gonadotropin blood level in a normal, healthy person from conception until death. During gestation, the time of greatest cell proliferation, the fetus is exposed to a high blood level of human chorionic gonadotropin (hCG) (a gonadotropin hormone that is present in significant amounts only during pregnancy and that has 83% sequence homology with LH and shares the same receptors as LH). (Fisher D A, Endocrinology of fetal development, in Williams Textbook of Endocrinology, edited by Wilson J D, Foster D W, Kronenberg H M, Larsen P R, W.B. Saunders Co., Philadelphia, Pa. (1998)). During gestation, the body also secretes a high blood level of activin, which also has been shown to increase cell proliferation in several tissues. (Qu J, Thomas K, Inhibin and activin production in human placenta, Endocrine Reviews 16:485-507 (1995)). Activin also stimulates the secretion of FSH and, to a lesser extent LH, from the pituitary gland. (Robertson D M, McLachlan R I, Burger H G, Inhibin-related proteins in the male, In The Testis, edited by Burger H, de Kretser D, Raven, New York, N.Y. (1989)).

As illustrated in FIG. 1, there is another peak in the blood level of LH and FSH during infancy, i.e. approximately the first year of life, another time of rapid cell proliferation, during which time a human normally doubles in mass. After the first year of life, the blood level of LH remains virtually undetectable and the blood level of FSH remains relatively low, as this is a period of relatively low mitogenicity. However, during the approximately five year period of puberty (around ages 13-18), another period of rapid cell proliferation, the blood level of LH and FSH gradually increases, as the body again almost doubles in mass. (Winter J S D, Faiman C, Reyes F I, Gonadotropins and steroid hormones in the blood and urine of prepubertal girls and other primates, Clin Endocrinol Metab 7:513-530 (1978)).

During or around the peak adult reproductive period (approximately ages 18-35), the blood level and activity of FSH and LH are elevated as compared to prepubertal childhood, but their mitogenic effects on the cell cycle are probably counteracted by the sex steroid hormones estrogen and testosterone. (Reichlin S, Neuroendocrinology, in Williams Textbook of Endocrinology, edited by Wilson J D, Foster D W, Kronenberg H M, Larsen P R, W.B. Saunders Co., Philadelphia, Pa., p. 212-213 (1998)). As shown in FIG. 1, the blood level of LH and FSH fluctuate in females according to the reproductive cycle. (Reame N, Saunder S E, Kelch R P, Pulsatile gonadotropin secretion during the menstrual cycle: evidence for altered frequency of gonadotropin-releasing hormone secretion, J Clin Endocrinol Metab 59:328-337 (1984)). During the adult reproductive period, the blood level, production, function or activity of activin also is counteracted by follistatin and/or inhibin. (Halvorson, L M & Chin W W, Gonadotropic hormones: biosynthesis, secretion, receptors, and action, in Reproductive Endocrinology, 4th ed., Yen, S S C, Jaffe R B & Barbieri R L, eds.: 94-97, W.B. Saunders, Philadelphia, Pa. (1999)).

The decline of reproductive function is accompanied by the onset and progression of senescence (also known as menopause in females and andropause in males). (Lamberts S W, van den Beld A W, van der Lely A J, The endocrinology of aging, Science, October 17; 278(5337):419-24 (1997)). As shown in FIG. 1, during senescence, the blood level of LH and FSH increases, sometimes reaching its highest level except for gestation during this period. (Yen S C C, The biology of menopause, J Reprod Med 18:287-296 (1977); Harman D M, Tsitouras P D, Reproductive hormones in aging men I: Measurement of sex steroids, basal luteinizing hormone, and Leydig cell response to human chorionic gonadotropin, J Clin Endocrinol Metab 51:35-40 (1980)). The increase in LH and FSH is much more rapid and sudden in females than in males. (Sherman B M, West J H, Korenman S G, The menopausal tradition: analysis of LH, FSH, estradiol and progesterone concentrations during menstrual cycles of older women, J Clin Endocrinol Metab 42:629-636 (1976)). One study has shown a three- to four-fold increase in LH serum concentrations and a four- to eighteen-fold increase in FSH serum concentrations in elderly women. (Chakravarti S, Collins W P, Forecast J D, Newton J R, Oram D H, Studd J W, Hormonal profiles after the menopause, Br Med J Oct. 2, 1976; 2(6039):784-7). Likewise, elderly men also experience a greater than two-fold, and three-fold, increase in LH and FSH serum concentrations, respectively. (Neaves et al. 1984). In addition, mRNA levels of gonadotropin releasing hormone in the hypothalamus of elderly women are increased. (Rance N E, Uswandi S V. Gonadotropin-releasing hormone gene expression is increased in the medial basal hypothalamus of postmenopausal women. Journal of Clinical Endocrinology and Metabolism. 81(10):3540-6 (1996)). This increased production of LH and FSH is due, at least in part, to decreased production of sex steroid hormones and of inhibin. (Yen S C C, The biology of menopause, J Reprod Med 18:287-296 (1977)). In elderly men, serum LH concentrations correlate much more closely with frailty than testosterone concentrations. (van den Beld A, Huhtaniemi I T, Pettersson K S, Pols H A, Grobbee D E, de Jong F H, Lamberts S W, Leutinizing hormone and different genetic variants as indicators of frailty in healthy elderly men, J Clin Endocrinol Metab April 1999; 84(4): 1334-9).

Methods and Agents for Slowing, Preventing or Delaying Senescence or Treating or Preventing Diseases Associated with Senescence

According to an aspect of the present invention, the increase in the blood level, production, function or activity of LH or FSH during senescence is associated with an upregulation of the cell cycle. Thus, an embodiment of the present invention encompasses slowing, preventing or delaying senescence or preventing or treating a disease associated with senescence by administering, to a subject, one or more LH/FSH-inhibiting agents (i.e., agents that decrease or regulate the blood level, production, function, or activity of LH or FSH).

Examples of LH/FSH-inhibiting agents include, but are not limited to, gonadotropin releasing hormone (GnRH) or GnRH analogs. GnRH and GnRH analogs can be administered to decrease or regulate the blood level, production, function, or activity of LH or FSH. Studies have shown that an increased levels of GnRH or GnRH analogs will result in significant decreases in LH and FSH levels. (Thorner M O, et al., The anterior pituitary, in Williams Textbook of Endocrinology 9th edition, eds. Wilson J D, Foster D W, Kronenberg H, Larsen P R, 269, W.B. Saunders Company, Philadelphia, Pa. (1998)). For example, leuprolide, a GnRH analog, has been shown to increase pituitary secretion of LH and FSH for several days after initial administration. (Mazzei T, et al., Pharmacokinetics, endocrine and antitumor effects of leuprolide depot (TAP-144-SR) in Advanced Prostatic Cancer: A Dose Response Evaluation, Drugs in Experimental and Clinical Research, 15:373-387 (1989)). Thereafter, pituitary GnRH receptors are down regulated, resulting in a significant decrease in LH and FSH secretion. (Mazzei T, et al., Human pharmacokinetic and pharmacodynamic profiles of leuprorelin acetate depot in prostatic cancer patients, Journal of Internal Medicine Research, 18(suppl):42-56 (1990)). Examples of GnRH analogs that are useful in the present invention include, but are not limited to, leuprolide, triptorelin, buserelin, nafarelin, desorelin, histrelin, and goserelin.

Additional examples of LH/FSH-inhibiting agents that may be administered in accordance with the present invention include, but are not limited to, inhibin or follistatin, or compounds that stimulate the production of inhibin or follistatin, which will inhibit FSH secretion, and to a lesser extent LH secretion. (Lee S, Rivier C. Effect of repeated activin-A treatment on the activity of the hypothalamic-pituitary-gonadal axis of the adult male rat, Biology of Reproduction, 56(4):969-75 (1997)). Inhibin and follistatin bind to and inactivate activin, which stimulates secretion from the pituitary of FSH, and to a lesser extent LH. (Robertson D M, et al., Inhibin-related proteins in the male, In The Testis, 2nd edition, eds. Burger H and deKretser D, 1989:231-254, Raven, New York (1989); Xiao S, et al., Interaction between activin and follicle-stimulating hormone-suppressing protein/follistatin in the regulation of basal inhibin production by cultured rat granulosa cells, Endocrinology, 131(5):2365-70 (1992)). By blocking the action of activin, inhibin and follistatin can decrease LH or FSH secretion.

Yet other examples of LH/FSH-inhibiting agents that may be administered in accordance with the present invention include, but are not limited to, a vaccine or antibody that stimulates the production of antibodies that inhibit the activity of LH, FSH, or GnRH. Additional examples of LH/FSH-inhibiting agents include, but are not limited to, a vaccine or antibody that stimulates the production of antibodies that block a LH receptor, a FSH receptor, or a GnRH receptor. Examples of such vaccines include, but are not limited to, the Talwar vaccine and the vaccine marketed under the trade name GONADIMMUNE® by Aphton Corporation.

Further examples of LH/FSH-inhibiting agents include, but are not limited to, a GnRH antagonist; a GnRH receptor blocker, such as citrorelix or abberelix; a compound that regulates expression of a LH or FSH receptor; and a compound that regulates post-receptor signaling of a LH or FSH receptor. Other examples of LH/FSH-inhibiting agents include, but are not limited to, physiologically acceptable analogs, metabolites, precursors and salts of any of the foregoing LH/FSH-inhibiting agents.

According to another aspect of the invention, during senescence, activin bioavailability increases, due, at least in part, to decreased levels or production of inhibin and/or follistatin. (Burger H G, Dudley E C, Hopper J L, Groome N, Guthrie J R, Green A, Dennerstein L, Prospectively measured levels of serum follicle-stimulating hormone, estradiol, and the dimeric inhibins during the menopausal transition in a population-based cohort of women, J Clin Endocrinol Metab November 84(11):4025-30 (1999)). Activin consists of dimers of beta subunits, designated by A, B, C, D, and E, yielding 32 different types of activin. In accordance with the present invention, and contrary to conventional teachings that activin-A inhibits cell proliferation, high concentrations of activin-A downregulate activin receptors and increase cell proliferation.

According to the present invention, an increase in the blood level, production, function or activity of activin during senescence is associated with an upregulation of the cell cycle. Accordingly, another embodiment of the present invention encompasses slowing, preventing or delaying senescence or preventing or treating a disease associated with senescence by administering, to a subject, one or more activin-inhibiting agents (i.e., agents that decrease or regulate the blood level, production, function or activity of activin).

Examples of activin-inhibiting agents include, but are not limited to, activin antagonists, such as inhibin or follistatin; compounds that stimulate the production of inhibin or follistatin; and compounds that bind to activin or to activin receptors on cells in order to block activin from binding to its receptors. Additional examples of activin-inhibiting agents encompassed by the present invention include, but are not limited to, activin receptor blockers, compounds that regulate expression of activin receptors and agents that regulate post-receptor signaling of activin receptors. Yet other examples of activin-inhibiting agents include, but are not limited to, vaccines or antibodies that stimulate the production of antibodies that recognize, bind to, or block or substantially reduce the activity of activin or one or more of activin's receptors. Other examples of activin-inhibiting agents include, but are not limited to, physiologically acceptable analogs, metabolites, precursors and salts of any of the aforementioned activin-inhibiting agents.

Also according to the present invention, a decrease in the blood level, production, function or activity of follistatin is associated with an upregulation of the cell cycle. Accordingly, another embodiment of the present invention encompasses slowing, preventing or delaying senescence or preventing or treating a disease associated with senescence by administering, to a subject, one or more follistatin-promoting agents (i.e., agents that increase or regulate blood level, production, function, or activity of follistatin).

Examples of follistatin-promoting agents include, but are not limited to, follistatin and compounds that stimulate production of follistatin. Other examples of follistatin-promoting agents include, but are not limited to, compounds that regulate expression of follistatin receptors and agents that regulate post-receptor signaling of follistatin receptors. Additional follistatin-promoting agents include, but are not limited to, physiologically acceptable analogs, metabolites, precursors and salts of any of the aforementioned follistatin-promoting agents, such as, for example, follistatin-related protein.

Additionally, according to the present invention, a decrease in the blood level, production, function or activity of inhibin also is associated with an upregulation of the cell cycle. Accordingly, another embodiment of the present invention encompasses slowing, preventing or delaying senescence or preventing or treating a disease associated with senescence by administering, to a subject, one or more inhibin-promoting agents (i.e., agents that increase or regulate blood level, production, function, or activity of inhibin).

Examples of inhibin-promoting agents include, but are not limited to, inhibin and agents that stimulate the production of inhibin. Other examples of inhibin-promoting agents include, but are not limited to, compounds that regulate expression of inhibin receptors and compounds that regulate post-receptor signaling of inhibin receptors. Additional examples of inhibin-promoting agents include, but are not limited to, analogs, metabolites, precursors and salts of any of the aforementioned inhibin-promoting agents.

The present invention further encompasses a method for inhibiting the rate of telomere shortening. During the cell cycle, the chromosomes are aligned at their ends by telomeres, which are necessary for completion of cell division. (Alberts B, Bray D, Lewis J, Raff M, Roberts K, Watson J D, The cell nucleus, in Molecular Biology of the Cell, Garland Publishing, Inc., New York, N.Y., p. 385-481 (1983)). In normal healthy cells, each time a cell divides, its daughter cells have shorter telomeres. (Saretzki G, Von Zglinicki T, Replicative aging, telomeres, and oxidative stress, Ann. N. Y. Acad. Sci. Apr; 959:24-9 (2002)). After a finite number of cell cycles, the telomeres become too short for a cell to divide and the cell eventually dies. (Tzukerman M, Selig S, Skorecki K, Telomeres and telomerase in human health and disease, J. Pediatr. Endocrinol. Metab., March; 15(3):229-40 (2002)). Progressive shortening of the telomeres leads to a disruption in the protein packaging on the end of the telomere and causes a growth-arrest response through DNA-damage recognition pathways. (Griffith J D, Comeau L, Rosenfield S, Stansel R M, Bianchi A, Moss H, de Lange T, Mammalian telomeres end in a large duplex loop, Cell 97:503-514 (1999)). In other words, the number of cells that constitutes the potential progeny of a normal, healthy parent cell is finite. Thus, “older” cells that are the product of many previous cell cycles have much shorter telomeres than “younger” cells that are the product of only a few cell cycles.

According to the present invention, an increased rate of telomere shortening is associated with an upregulation of the cell cycle. Accordingly, the present invention also encompasses inhibiting or slowing the rate of telomere shortening by administering, to a subject, one or more of the aforementioned LH/FSH-inhibiting agents, activin-inhibiting agents, inhibin-promoting agents, or follistatin-promoting agents, including, but not limited to, analogs, metabolites, precursors, or salts thereof. In another embodiment of the invention, the progression of senescence in a tissue can be quantified by taking periodic biopsy samples of the tissue and measuring the average length of the telomeres of cells in the sample.

In another embodiment, the present invention encompasses a method of decreasing or regulating a subject's mitogenic index. The mitogenic index measures the rate of cell proliferation in the subject, compared to the rate of cell proliferation in a control cell line. The mitogenic index correlates to the subject's rate of senescence or propensity for diseases associated with senescence. For example, the present invention encompasses the following method and system for measuring the mitogenic index of a subject. First, a test sample comprising a first plurality of cells from a standardized cell line (e.g., human fibroblast cells, human neuroblastoma cells) and a control sample of the standardized cell line each are cultured in a standard growth medium (e.g., agar). Next, a tissue collecting means, such as a needle, is used to collect a tissue sample, such as serum, plasma or cerebrospinal fluid, from the subject. In an embodiment, a blood sample is taken from the subject and centrifuged to separate a serum sample, which may contain LH, FSH, activin, inhibin and/or follistatin. Because LH and FSH are secreted in a pulsatile fashion, in an embodiment, several serum samples are taken over the course of a few hours and the serum samples are mixed into a averaged tissue sample. An adding means, such as a pipette, is used to add the tissue sample to the test sample, producing a combined sample. The cells in the combined sample and in the control sample are allowed to cycle for a predetermined time, such as twenty four hours. After this time, proliferation of cells in the combined sample and in the control sample is measured using a measuring means, such as, for example, BrdU labeling, thymidine labeling, or a cell counter. The mitogenic index of the subject is then computed using a computing means, such as a computer, to calculate the ratio of the number of cells (or rate of proliferation) in the combined sample to the number of cells (or rate of proliferation) in the control sample.

If the subject has high blood level, production, function or activity of LH, FSH, and/or activin and/or low blood level, production, function or activity of inhibin or follistatin, the subject's serum is expected to cause a high rate of proliferation of the test sample, resulting in a high mitogenic index, and thus a high rate of senescence. Accordingly, the present invention encompasses administering one or more of the aforementioned LH/FSH-inhibiting agents, activin-inhibiting agents, inhibin-promoting-agents, and follistatin-promoting agents, including analogs, metabolites, precursors, and salts thereof, in order to decrease or regulate the subject's mitogenic index.

In other embodiments of the invention, a sex steroid hormone, such as estrogen, progesterone, or testosterone, or an analog, metabolite, precursor, or salt thereof, may be co-administered with an LH/FSH-inhibiting agent, activin-inhibiting agent, inhibin-promoting agent, or follistatin-promoting agent, including those identified above. Through a negative feedback loop, the presence of estrogen, progesterone, or testosterone signals the hypothalamus to decrease the secretion of GnRH. (Gharib S D, et al., Molecular biology of the pituitary gonadotropins, Endocrine Reviews, 11:177-199 (1990); Steiner R A, et al., Regulation of leutinizing hormone pulse frequency and amplitude by testosterone in the adult male rat, Endocrinology, 111:2055-2061 (1982)). The subsequent decrease in GnRH decreases the secretion of LH and FSH. (Thomer M O, et al., The anterior pituitary, in Williams Textbook of Endocrinology, 9th edition, eds. Wilson J D, Foster D W, Kronenberg H, Larsen P R, 269, W.B. Saunders Company, Philadelphia, Pa. (1998)). Thus, according to the present invention, co-administration of estrogen, progesterone or testosterone further decreases secretion of LH or FSH, and thereby inhibits upregulation of the cell cycle, sometimes with synergistic effects. Moreover, because administration of the LH/FSH-inhibiting agents described above may have the undesired side-effect of reducing the natural production of sex steroids, the present invention also encompasses co-administration of sex steroids in order to replenish the sex steroids.

In another embodiment, the present invention encompasses slowing, preventing or delaying senescence, or treating or preventing atherosclerosis, osteoporosis, or brain damage associated with acute brain injury, by administering a cell cycle inhibitor (i.e., an agent that inhibits upregulation of the cell cycle or cell cycling). An example of such a cell cycle inhibitor includes low density lipoprotein receptor related protein receptor associated protein (“RAP”). RAP binds to and inactivates alpha-2 macroglobulin (“A2M”) receptors preventing the binding of A2M, which has been shown to bind to activin. According to the present invention, the A2M:activin complex binds to the A2M receptor in order to mediate some of activin's activity, and because activin has been shown to increase cell proliferation, RAP can be used in accordance with the present invention.

Another example of a cell cycle inhibitor is a vaccine or antibody against proteins involved in promoting cell division (e.g. cell cycle proteins such as CDKs). Although it takes approximately ten days for the body to produce antibodies after administration of a vaccine or antibody, passive immunization with antibodies to each of these cell cycle proteins should immediately decrease their serum levels.

Yet another example of a cell cycle inhibitor is taxol, which inhibits cell division by blocking changes in microtubules and the cytoskeleton. Other examples of cell cycle inhibitors encompassed by the present invention include, but are not limited to, vitamin A (i.e., retinoic acid), hydroxyurea, colchicines, and cholesterol lowering drugs, such as lovastatin and provastatin.

Treatment Targets

The present invention encompasses slowing, preventing or delaying senescence, or treating or preventing a disease associated with senescence, or inhibiting or preventing upregulation of the cell cycle, or decreasing the mitogenic index, or inhibiting the shortening of telomeres, by administering one or more of the aforementioned LH/FSH-inhibiting agents in combinations, quantities and dosage regimens in order to decrease or regulate the blood level, production, function or activity of LH or FSH to be at or near one of the following target blood levels, target productions, target functions or target activities of LH and FSH.

In one embodiment, the target blood level, target production, target function, or target activity of LH or FSH is that occurring at or near the time of greatest reproductive function, which in humans corresponds to 18 to 35 years of age. For example, a normal blood level of LH around this time is approximately 0-10.0 mIU/mL for males and approximately 0.4-92.9 mIU/mL for females (which fluctuates with reproductive cycle). A normal blood level of FSH around this time is approximately 2.0-22.6 mIU/mL for males and approximately 2.9-29.5 mIU/mL for females (which also fluctuates with reproductive cycle). In another embodiment, the target blood level, target production, target function, or target activity of LH or FSH is that which is undetectable or nearly undetectable by conventional means known in the art. For example, a blood level of 0.7 mIU/mL for both LH and FSH is currently undetectable in a clinical laboratory. In another embodiment of the invention, the target blood level, target production, target function, or target activity of LH or FSH is as low as possible without unacceptable adverse side effects. An unacceptable adverse side effect is an adverse side effect that, in the reasonable judgment of one of ordinary skill in the art, has costs that outweigh the benefits of treatment.

It will be apparent to one of ordinary skill in the art, in light of this specification, that the subject's blood level, production, function, or activity of LH or FSH may be periodically monitored and the combinations, quantities, and dosage regimens of the LH/FSH-inhibiting agents may be titrated or varied in order to achieve the target blood level, target production, target function or target activity of LH and FSH. In an embodiment, the dosage for a LH/FSH-inhibiting agent, for example leuprolide acetate, may be between approximately 0.01 mcg/kg/hour and approximately 100 mg/kg/day. Such an LH/FSH-inhibiting agent may be administered, for example, as an hourly subcutaneous injection, or as a constant rate intravenous infusion for a number of hours, or as a monthly or semi-monthly intramuscular injection of the agent in a time released form (such as an agent encased in a polymer matrix or microspheres), or using other dosage forms or schedules that will be apparent to one of ordinary skill in the art, in light of this specification. In such an embodiment, the subject may initially be administered a low dose, for example approximately 0.01 mcg/kg/hour. After approximately two weeks, LH and FSH blood levels may be measured. If LH and FSH bloods levels are still higher than the target, then the dose gradually may be increased (for example by 0.1 mcg/kg/hour). This titration can be repeated until the blood level, production, function or activity of LH or FSH reaches the desired target blood level, target production, target function, or target activity for LH or FSH, as set forth above.

For example, a 30 mg time-released dose of leuprolide acetate was administered to an approximately 72-year old male. The leuprolide acetate was encased in a polymer matrix so that it would be gradually released over approximately four months. After a period of two weeks, the subject's blood level of LH was undetectable and the subject's blood level of FSH was approximately 5 mIU/mL. In another example, a dose of 1.88 mg of leuprolide acetate in a polymer matrix, gradually released over approximately one month, is expected to reduce LH and FSH blood levels to undetectable levels in many subjects. It will be apparent to one of ordinary skill in the art, in light of this specification, that in order to achieve one of these targets, the dosage of the LH/FSH-inhibiting agent will vary from subject to subject in light of factors such as age, gender, body weight, diet, the disease being treated, the progression of the disease, and other drugs being administered.

The present invention further encompasses slowing, preventing or delaying senescence, or treating or preventing a disease associated with senescence, or inhibiting or preventing upregulation of the cell cycle, or decreasing the mitogenic index, or inhibiting the shortening of telomeres, by administering one of the aforementioned activin-inhibiting agents in combinations, quantities and dosage regimens in order to decrease or regulate the blood level, production, function or activity of activin to be at or near one of the following target blood levels, target productions, target functions or target activities of activin.

In an embodiment, the target blood level, target production, target function or a target activity of activin is that occurring at or near the time of greatest reproductive function. For example, the normal blood level of activin-A around this time is approximately 590 pg/mL in both men and women. In another embodiment, the target blood level, target production, target function or target activity of activin is that which is undetectable or nearly undetectable by conventional means known in the art. In yet another embodiment, the target blood level, target production, target function or target activity of activin is approximately as low as possible without unacceptable adverse side effects.

It will be apparent to one of ordinary skill in the art, in light of this specification, that a subject's blood level, production, function or activity of activin may be periodically monitored and the combinations, quantities, and dosage regimens of the activin-inhibiting agents may be titrated or varied in order to achieve the target blood level, target production, target function or target activity of activin. In an embodiment, the dosage of an activin-inhibiting agent may be between approximately 0.01 mcg/kg/hour and approximately 100 mg/kg/day. Such an activin-inhibiting agent may be administered, for example, as an hourly subcutaneous injection, or as a constant rate intravenous infusion for a number of hours, or as a monthly or semi-monthly intramuscular injection of the agent in a time released form (such as an agent encased in a polymer matrix or microspheres), or using other dosage forms or schedules that will be apparent to one of ordinary skill in the art in light of this specification. In such an embodiment, the subject may first be administered approximately 0.01 mcg/kg/hour of the activin-inhibiting agent. After approximately two weeks, the activin blood level could be measured, and the dose adjusted based on the blood level. For example, if the blood level is lower than the target, the dosage could be gradually increased (for example in 0.1 mcg/kg/hour increments every two weeks) until the activin blood level reaches the desired target, as set forth above. In another example, dosages for follistatin, an activin-inhibiting agent and a follistatin-promoting agent, are discussed below with respect to the follistatin promoting-agents. It will be apparent to one of ordinary skill in the art, in light of this specification, that in order to achieve one of these targets, the dosage of the activin-inhibiting agent will vary from subject to subject in light of factors such as age, gender, body weight, diet, the disease being treated, the progression of the disease, and other drugs being administered.

The present invention also encompasses slowing, preventing or delaying senescence, or treating or preventing a disease associated with senescence, or inhibiting or preventing upregulation of the cell cycle, or decreasing the mitogenic index, or inhibiting the shortening of telomeres, by administering one or more of the aforementioned follistatin-promoting agents in combinations, quantities and dosage regimens in order to increase or regulate a blood level, production, function or activity of follistatin to be approximately as high as possible without unacceptable adverse side effects.

It will be apparent to one of ordinary skill in the art, in light of this specification, that a subject's blood level, production, function, or activity of follistatin may be periodically monitored and the combinations, quantities, and dosage regimens of the follistatin-promoting agents may be titrated or varied in order to achieve this target blood level, target production, target function or target activity of follistatin. The dosage for a follistatin-promoting agent, such as follistatin, may be, for example, between approximately 0.01 mcg/kg/hour and approximately 100 mg/kg/day. Such a follistatin-promoting agent may be administered, for example, as an hourly subcutaneous injection, or as a constant rate intravenous infusion for a number of hours, or as a monthly or semi-monthly intramuscular injection of the agent in a time released form (such as an agent encased in a polymer matrix or microspheres), or using other dosage forms or schedules that will be apparent to one of ordinary skill in the art in light of this specification.

For example, the normal steady state circulating blood level of follistatin remains relatively constant in adulthood at approximately 6.6±0.3 ng/mL for women and approximately 5.4±0.2 ng/mL for men. (Kettel M et al., Circulating levels of follistatin from puberty to menopause, Fertil. Steril., March 1996; 65(3):472-6). According to the present invention, follistatin administered by a constant rate intravenous infusion (such as by an implantable pump), is expected to increase the steady state circulating blood level of follistatin as follows:

| | | Expected increase in |
|---|---|---|
| | Dose in 24-hr | steady state |
| Follistatin Infusion Rate | per 70 kg subject | follistatin blood level |
| (mcg/hr/kg) | (mg) | (ng/mL) |
| 1.0 | 1.68 | 0.54 |
| 5.0 | 8.40 | 2.70 |
| 10.0 | 16.8 | 5.40 |
| 25.0 | 42.0 | 13.5 |
| 50.0 | 84.0 | 27.0 |
| 100.0 | 168.0 | 54.0 |

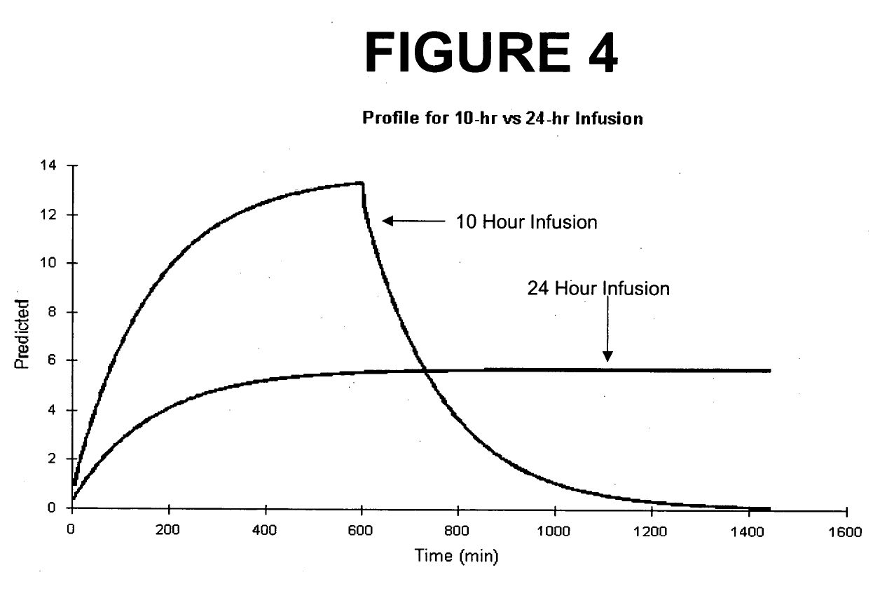

At the end of an infusion, the blood level of follistatin is expected to decrease to a normal blood level with a half-life of approximately 130 minutes. FIG. 4 illustrates the expected blood level of follistatin using a 10-hour constant rate intravenous infusion of approximately 10 mcg/kg/hour of follistatin and a 24 constant rate intravenous infusion of approximately 10 mcg/kg/hour of follistatin.

Accordingly, for example, a 70 kg subject could initially be administered 10 mcg/kg/hour via an implantable pump providing a 24 hour constant rate infusion (or a total of approximately 16.8 mg per day via another route, such as subcutaneous injection). After approximately two weeks, the follistatin blood level or the activin blood level, or the subject's mitogenic index, could be monitored. If, for example, the side effects are minimal and the follistatin level is lower than the target or the activin level is higher than the target or the mitogenic index is higher than the target, then the dose of follistatin could be increased. If, for example, the side effects are too great, the dose of the follistatin could be decreased. It will be apparent to one of ordinary skill in the art, in light of this specification, that the dosage of the follistatin-promoting agent will vary from subject to subject in light of factors such as age, gender, body weight, diet, the disease being treated, the progression of the disease, state, and other drugs being administered.

The present invention further encompasses slowing, preventing or delaying senescence, or treating or preventing a disease associated with senescence, or inhibiting or preventing upregulation of the cell cycle, or decreasing the mitogenic index, or inhibiting the shortening of telomeres, by administering the aforementioned inhibin-promoting agents in combinations, quantities and dosage regimens in order to increase or regulate the blood level, production, function or activity of inhibin to be at or near one of the following target blood levels, target productions, target functions or target activities of inhibin.

In an embodiment of the invention, the target blood level, target production, target function, or target activity of inhibin is that occurring at or near the time of greatest reproductive function of the subject. For example, the normal blood level of inhibin at or around this time is approximately 300-1000 mIU/mL for women (which varies with the reproductive cycle) and approximately 232-866 mIU/mL for men. (Halvorson L M, DeCherney A H, Inhibin, activin, and follistatin in reproductive medicine, Fertility and Sterility, 65(6), March 1996). In another embodiment of the invention, the target blood level, target production, target function, or target activity of inhibin is approximately as high as possible without unacceptable adverse side effects.

It will be apparent to one of ordinary skill in the art, in light of this specification, that the subject's blood level, production, function, or activity of inhibin may be periodically monitored and the combinations, quantities, and dosage regimens of the inhibin-promoting agents may be titrated or varied in order to achieve the target blood level, target production, target function or target activity of inhibin. For example, the dosage for an inhibin-promoting agent, such as inhibin itself, may be between approximately 0.01 mcg/kg/hour and approximately 100 mg/kg/day. In such an embodiment, the subject will first be administered approximately 0.01 mcg/kg/hour of an inhibin-promoting agent. Such a follistatin-promoting agent may be administered, for example, as an hourly subcutaneous injection, or as a constant rate intravenous infusion for a number of hours, or as a monthly or semi-monthly intramuscular injection of the agent in a time released form (such as an agent encased in a polymer matrix or microspheres), or using other dosage forms or schedules that will be apparent to one of ordinary skill in the art in light of this specification. In such an embodiment, after approximately two weeks, an inhibin blood level will be measured. If the desired target has not been reached, and there are no unacceptable side effects, the dose will gradually be increased (for example in 0.1 mcg/kg/hour increments) until the blood level of inhibin reaches the desired target blood level, as set forth above. It will be apparent to one of ordinary skill in the art, in light of this specification, that in order to achieve the targets for inhibin set forth above, the dosage of the inhibin-promoting agent will vary from subject to subject in light of factors such as age, gender, body weight, diet, the disease being treated, the progression of the disease, and other drugs being administered.

The present invention further encompasses administering two or more of the LH/FSH-inhibiting agents, activin-inhibiting agents, follistatin-promoting agents, or inhibin-promoting agents in order to achieve the target blood level, target production, target activity, and target function for one or more of LH, FSH, activin, follistatin and inhibin. For example, leuprolide acetate (an LH/FSH-inhibiting agent) may be administered in tandem with follistatin (an activin-inhibiting agent and a follistatin-promoting agent) both to decrease the blood levels of LH, FSH, and activin and to increase the blood levels of follistatin. In yet another embodiment the present invention further encompasses administering one or more of the LH/FSH-inhibiting agents, activin-inhibiting agents, follistatin-promoting agents, or inhibin-promoting agents in order to regulate a ratio of a blood level, production, function and activity of two or more of LH, FSH, activin, follistatin, and inhibin.

In embodiments of the present invention, the blood level, production, function or activity of LH or FSH, or the blood level, production, function or activity of activin are continuously decreased or regulated, or the blood level, production, function, or activity of inhibin or follistatin are continuously increased or regulated, by monitoring the blood level, production, function or activity of LH, FSH, activin, inhibin, and/or follistatin and making adjustments to amounts or types of the agent or agents being administered via a feedback control system.

According to embodiments of the present invention, administration of LH/FSH-inhibiting agents, activin-inhibiting-agents, inhibin-promoting agents, follistatin-promoting agents, sex steroids, or cell cycle inhibitors listed above, can be oral, by injection, by constant rate infusion, by inhalation, by patch, intrathecally (i.e., into the arachnoid membrane of the brain or spinal cord), by a time release pump, by a time-release injection (such as an agent encased in microspheres or a polymer matrix) or by other effective means. According to other embodiments of the invention, administration of LH/FSH-inhibiting agents, activin-inhibiting agents, inhibin-promoting agents, follistatin-promoting agents, or sex steroids, including those identified above, can be in a single dose, multiple doses, in a sustained release dosage form, in a pulsatile form, or in any other appropriate dosage form or amount. Early administration is preferred, as the sooner upregulation of the cell cycle is inhibited, the slower the progression of senescence or diseases associated with senescence. The duration of treatment could range from a few days or weeks to the remainder of the patient's life.

Examples of Treating or Preventing Diseases Associated with Senescence

The following examples of using the present invention to treat particular diseases associated with senescence are presented for illustration purposes only and in no way limit the invention to the treatment or prevention of the diseases enumerated herein. The present invention may be used to treat any disease associated with senescence, including, but not limited to, those diseases listed above.

1. Atherosclerosis

The present invention encompasses treating or preventing atherosclerosis, a disease associated with senescence. Atherosclerosis is a progressive disease process of arterial tissues that is a principal contributor to the pathogenesis of myocardial and cerebral infarction, gangrene, and loss of function in the extremities. (Lusis A J, Atherosclerosis, Nature, 407:233-241 (Sep. 14, 2000)). The disease initiates spontaneously or from an injury to the tissue on the interior of an arterial wall, especially at a branch point in the arteries. (Mora R, Lupu F, Simionescu N, Prelesional events in atherogenesis, Colocalization of apolipoprotein B, unesterified cholesterol and extracellular phospholipid liposomes in the aorta of hyperlipidemic rabbit, Atherosclerosis, October; 67(2-3):143-54 (1987)). Arterial injury may result from numerous sources, including but not limited to, physical trauma, including mild trauma associated with the normal function of the tissue, for example contraction of smooth muscle within the arterial wall or sheer forces from normal blood flow. (Ross R, The pathogenesis of atherosclerosis—an update, N Engl J Med., Feb. 20; 314(8):488-500 (1986)). The sources of arterial injury, or increased susceptibility to arterial injury, are chronic in nature and thus progression of atherosclerosis is usually continuous without intervention. (Stary H C, The sequence of cell and matrix changes in atherosclerotic lesions of coronary arteries in the first forty years of life, Eur Heart J August; 11Suppl E:3-19 (1990)). Although atherosclerotic changes may begin in childhood, with increased worsening in the third decade of life, the pathologic manifestations of the disease become a major health concern as age increases into the fifth and sixth decade. Risk factors for atherosclerosis include, but are not limited to, genetic predisposition, hypercholesterolemia, hypertension, cigarette smoking, diabetes and obesity. (Lusis, A J (2000)).

The earliest detectable atherosclerotic lesion is called a “fatty streak,” which is associated with the progression of monocytes across the endothelial cell layer into the intima, the innermost layer of the arterial wall, at the site of arterial injury. In the intima, the monocytes are converted to macrophages, which become engorged with cholesterol and are also known as “foam cells.” In time, the foam cells die, contributing their cholesterol to the necrotic core of a lesion.

Some fatty streaks accumulate vascular smooth muscle cells, which migrate to the site of the fatty streak from the medial layer of the arterial wall. The lesions grow inward toward the wall and then outward into the lumen, the space in which blood flows through the artery. In some cases, the growth of the lesion is contributed to by the accumulation of lymphocytes. The growth of the lesion is accompanied by the proliferation of the monocytes, macrophages, vascular smooth muscle cells, endothelial cells, fibroblasts and/or lymphocytes. The lesion also accumulates lipoproteins and cholesterol and forms an extracellular connective tissue matrix. A well-developed atherosclerotic lesion is also known as a fibrous plaque, which can rupture, causing sudden occlusion of the artery by thrombus formation (e.g., a myocardial infarction).

In accordance with the present invention, and contrary to conventional teachings, increases in the blood level, production, activity or function of LH or FSH, often coinciding with increased age, contribute to atherosclerosis by causing an upregulation of the cell cycle and stimulating the increased proliferation of monocytes, macrophages, smooth muscle cells, or lymphocytes. For example, LH receptors are expressed in lymphocytes. (E.g., Lin J, et al., Lymphocytes from pregnant women express human chorionic gonadotropin/leutinizing hormone receptor gene, Mol Cell Endocrinol. Apr. 28; 111(1):R13-7 (1995)). Studies suggest that an increase in the production or activity of LH and FSH stimulates cell proliferation in lymphocytes. (Athreya B H, Pletcher J, Zulian F, Weiner D B, Williams W V, Subset-specific effects of sex hormones and pituitary gonadotropins on human lymphocyte proliferation in vitro, Clin Immunol Immunopathol March; 66(3):201-11(1993)). Accordingly, one aspect of the present invention encompasses preventing or treating atherosclerosis, or preventing or slowing proliferation of monocytes, macrophages, smooth muscle cells, endothelial cells, fibroblasts or lymphocytes, by administering one or more LH/FSH-inhibiting agents, including those identified above, that decrease or regulate the blood level, production, function or activity or LH or FSH.

Also in accordance with the present invention, and contrary to conventional teachings, increases in the blood level, production, function or activity of activin, or decreases in the blood level, production, function, or activity of inhibin or follistatin, are associated with stimulating increased proliferation of monocytes, macrophages, smooth muscle cells, endothelial cells, fibroblasts or lymphocytes. Accordingly, the present invention also encompasses preventing or treating atherosclerosis, or preventing or slowing proliferation of monocytes, macrophages, smooth muscle cells, endothelial cells, fibroblasts or lymphocytes, by administering one or more activin-inhibiting agents, including those identified above, that decrease the blood level, production, function or activity of activin. The present invention further encompasses preventing or treating atherosclerosis, or preventing or slowing proliferation of monocytes, macrophages, smooth muscle cells, endothelial cells, fibroblasts or lymphocytes, by administering one or more inhibin-promoting agents or follistatin-promoting agents, including those identified above, that increase the blood level, production, function or activity of inhibin or follistatin.

The present invention further encompasses a method of preventing or treating atherosclerosis, or preventing or slowing proliferation of monocytes, macrophages, smooth muscle cells, endothelial cells, fibroblasts, or lymphocytes by administering one of the aforementioned cell cycle inhibitors that prevent or inhibit cells from entering into the cell cycle. Such agents include, but are not limited to, low density lipoprotein receptor related protein receptor associated protein (“RAP”); a vaccine or antibody against proteins involved in promoting cell division (e.g. against cell cycle proteins such as CDK); taxol; vitamin A; hydroxyurea; colchicines; cholesterol lowering drugs, such as lovastatin or provastatin; and analogs, metabolites, precursors, and salts thereof.

2. Brain Cancer

The present invention further encompasses preventing or treating a brain cancer, a disease associated with senescence. By “brain cancer” is meant any abnormally increased proliferation of any type of neuronal cells. Examples of brain cancers include, but are not limited to, neuroma, anaplastic astrocytoma, neuroblastoma, glioma, glioblastoma multiforme, astrocytoma, meningioma, pituitary adenoma, primary CNS lymphoma, medulloblastoma, ependymoma, sarcoma, oligodendroglioma, medulloblastoma, spinal cord tumor, and schwannoma. (Hill J R, Kuriyama N, Kuriyama H, Israel M A, Molecular genetics of brain tumors, Arch Neurol April; 56(4):439-41 (1999)).

Most neuronal cells—that is cells that comprise or are found in the central nervous system, including, for example, neurons, microglia, and astrocytes—are “terminally differentiated,” meaning that they no longer possess the ability to complete the cell cycle. (Jacobsen M, Histogenesis and morphogenesis of cortical structures, in Developmental Neurobiology, M. Jacobsen, ed., Plenum, New York, N.Y., 1991, pp. 401-451). Although terminally differentiated neuronal cells may be able to enter the cell cycle, they are unable to complete the process and usually undergo apoptosis (i.e., cell death). (Multani A S, Ozen M, Narayan S, Kumar V, Chandra J, McConkey D J, Newman R A, Pathak S, Caspase-dependent apoptosis induced by telomere cleavage and TRF2 loss, Neoplasia Jul-Aug; 2(4):339-45 (2000)). Brain cancers may result when terminally differentiated neuronal cells lose the protective ability to apoptose and are able to complete the cell cycle, resulting in abnormally increased cell proliferation. (Hahn W C, Meyerson M, Telomerase activation, cellular immortalization and cancer, Ann Med March; 33(2):123-9 (2001)).

According to the present invention, an upregulation in the cell cycle, caused by increased mitogenic stimulus, contributes to the development of brain cancers by causing abnormally increased proliferation of neuronal cells that have lost the ability to apoptose. For purposes of this embodiment of the invention, “abnormally increased proliferation” means the increased proliferation of neuronal cells that interferes with the normal function of the central nervous system and/or threatens the life or health of the subject.

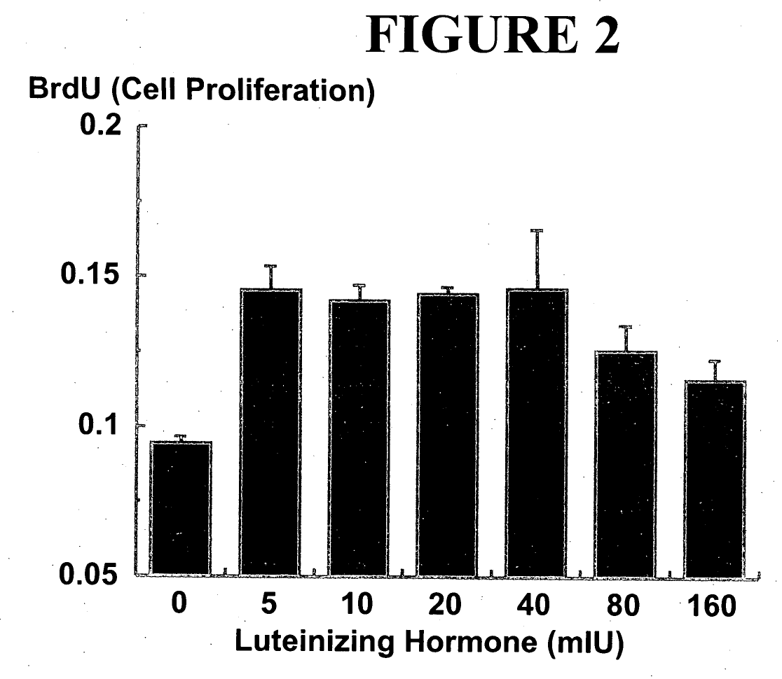

In accordance with the present invention, and contrary to conventional teachings, abnormally increased proliferation of neuronal cells is caused, at least in part, by an increase in the blood level, production, function or activity of LH or FSH. For example, a study was conducted to confirm that the presence of LH in neuroblastoma cells (i.e., neuronal tumor cells) stimulates cell proliferation. In that study, various amounts of LH, ranging from 0 to 160 mIU, were added to samples of neuroblastoma cells cultured in serum free media. The cultures were BrdU labeled to indicate the amount of cell division. As shown in FIG. 2, those cultures that received non-zero amounts of LH had a significantly increased rates of cell division as compared to cells that received no LH, with the highest rates occurring at LH concentrations of 5-40 mIU. Cells that received physiological concentrations of LH (5-10 mIU/ml) had a rate of cell proliferation approximately 50% higher than cells that received no LH.

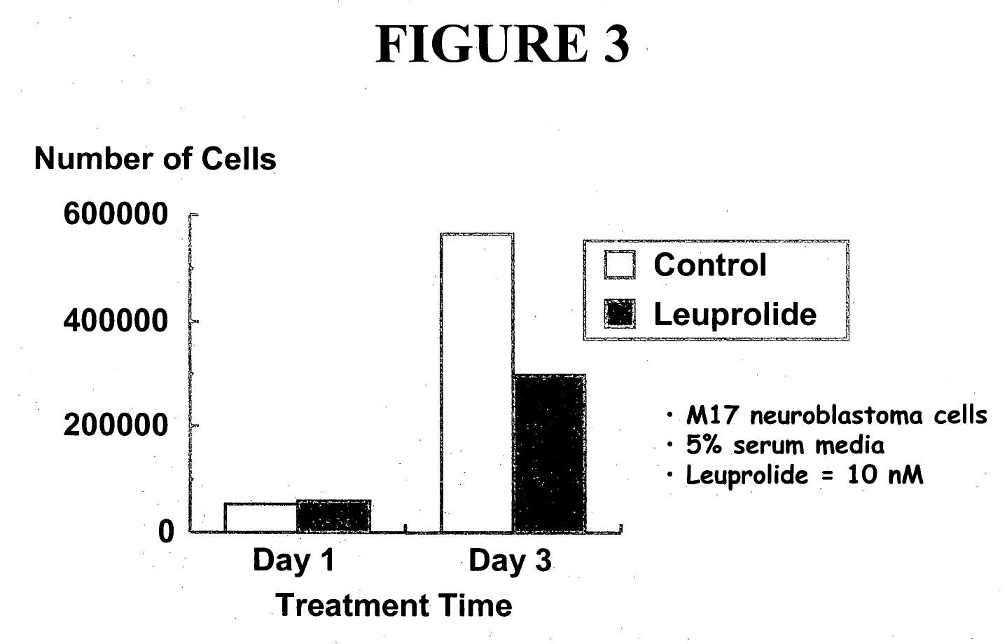

Thus, the present invention encompasses preventing or treating brain cancer, or preventing or slowing proliferation of neuronal cells, by administering one or more LH/FSH-inhibiting agents, including those identified above, that decrease or regulate the levels, production, function, or activity of LH or FSH. For example, a study was conducted to confirm that the administration to neuroblastoma cells of leuprolide, a GnRH analog that decreases the level, production, function or activity of LH and FSH, decreases proliferation of those cells. FIG. 3 illustrates the results for neuroblastoma cells exposed, in vitro, to leuprolide at a concentration of about 10 nM, which is approximately equivalent to a therapeutically effective blood level of leuprolide, according to the present invention. As shown in FIG. 3, after three days, neuroblastoma cells that received leuprolide had almost three-times less cell proliferation than neuroblastoma cells that received no leuprolide.

Also in accordance with the present invention, and contrary to conventional teachings, increased blood level, production, function or activity of activin or decreased levels, production, function, or activity of inhibin or follistatin is associated with stimulating abnormally increased proliferation of neuronal cells, leading to brain cancers. Accordingly, the present invention also encompasses treating or preventing brain cancers, or preventing or slowing proliferation of neuronal cells, by administering one or more activin-inhibiting agents, including those identified above, that decrease the blood level, production, function or activity of activin, or administering one or more inhibin-promoting agents or follistatin-promoting agents, including those identified above, that increase the blood level, production, function or activity of inhibin or follistatin.

3. Colorectal Cancer

The present invention also encompasses treating or preventing colorectal cancer, a disease associated with senescence. Colorectal cancer is the third most prevalent carcinoma and the second most frequent cause of cancer-related death in the United States, with 135,000 new diagnoses and 70,000 deaths each year. (Greenlee R T, Hill-Harmon M B, Murray T, Thun M, Cancer Statistics 2001, CA Cancer J Clin 51:15-36 (2001)) Evidence suggests that most colorectal cancers evolve through the formation of polyps, or small growths, in colorectal tissue, caused by abnormally increased proliferation of cells in colorectal tissue. (Robbins S L, Cotran R S, Kumar V, The gastrointestinal tract, in Pathologic Basis of Disease, edited by Robbins S L, Cotran R S, Kumar V. p. 797-883, 1984). (Farraye F A, Wallace M, Clinical significance of small polyps found during screening with flexible sigmoidoscopy, Gastrointest Endosc Clin N Am 12:41-51 (2002)) The incidence of this disease increases with age, with the highest incidence occurring during the fifth through seventh decades of life (Okamoto M, Shiratori Y, Yamaji Y, Kato J, Ikenoue T, Togo G, Yoshida H, Kawabe T, Omata M, Relationship between age and site of colorectal cancer based on colonoscopy findings, Gastrointest Endosc 55:548-51 (2002)).

According to the present invention, an upregulation of the cell cycle contributes to the formation of polyps in the colon and to colorectal cancer by causing abnormally increased proliferation of cells of colorectal tissue. For purposes of this embodiment of the invention, “abnormally increased proliferation” means an increased proliferation of cells that interferes with the normal function of the colorectal system and/or threatens the life or health of the subject.

In accordance with the present invention, and contrary to conventional teachings, abnormally increased proliferation of cells in colorectal tissue is mediated, at least in part, by age-related increases in the levels, production, activity or function of LH or FSH. For example, research has shown that the intestines of senescent rats, which have increased levels of LH and FSH, have an increased rate of cell proliferation. (E.g., Holt P R, Yeh K Y, Kotler D P, Altered controls of proliferation in proximal small intestine of the senescent rat, Proc Natl Sci USA Apr; 85(8):2771-5 (1988); Descner E E, Cell proliferation and colonic neoplasia, Scand J Gastroenterol Suppl 151:94-7 (1988)). In addition, in aging women, hormone replacement therapy (HRT), which indirectly lowers production of LH and FSH, has proven to be protective for colon cancer. (Jagadeesan U B, An incentive to start hormone replacement: the effect of postmenopausal hormone replacement therapy on the risk of colorectal cancer, J Am Geriatr Soc. 50:768-70 (2002)). In one study of 815 aging women, those who used HRT had a 40% lower probability of dying from colorectal cancer than those women who did not use HRT and those women who used HRT for four or more years had the lowest risk of colorectal cancer death. (Slattery M L, Anderson K, Samowitz W, Edwards S L, Curtin K, Caan B, Potter J D, Hormone replacement therapy and improved survival among postmenopausal women diagnosed with colon cancer (USA), Cancer Causes Control 10:467-73 (1999)).

Accordingly, the present invention encompasses treating or preventing colorectal cancer, or preventing or slowing colorectal polyp formation, or preventing or slowing proliferation of cells of colorectal tissue, by administering one or more LH/FSH-inhibiting agents, including those identified above, that decrease or regulate the blood level, production, function or activity of LH or FSH.

Also in accordance with the present invention, and contrary to conventional teachings, increased blood level, production, function or activity of activin and/or decreased levels, production, function, or activity of inhibin or follistatin is associated with stimulating abnormally increased proliferation of cells of colorectal tissue, which leads to the development of polyps and/or colorectal cancer. Accordingly, the present invention also encompasses treating or preventing colorectal cancer, or preventing or slowing colorectal polyp formation, or preventing or slowing proliferation of cells of colorectal tissue, by administering one or more activin-inhibiting agents, including those identified above, that decrease the blood level, production, function or activity of activin, or administering one or more inhibin-promoting or follistatin-promoting agents, including those identified above, that increase the levels, production, function or activity of inhibin or follistatin.

4. Myeloproliferative Disease

The present invention further encompasses preventing or treating a myeloproliferative disease, a disease associated with senescence. Myeloproliferative disease is a disease caused by, linked to, or otherwise associated with an upregulation of the cell cycle, contributing to an abnormally increased proliferation of myelogenous cells. Myelogenous cells are any cells that are derived from the bone marrow. For purposes of this embodiment of the invention, “abnormally increased proliferation” means proliferation of myelogenous cells that interferes with the normal function of the bone marrow and/or that threatens the life or health of the individual possessing myelogenous cells that exhibit this type of proliferation.

Examples of myeloproliferative diseases include, but are not limited to, Hodgkin's disease, multiple myeloma, lymphoma, transient myeloproliferative disorder (TMD) (also known as transient myeloproliferative syndrome), congenital transient leukemia, congenital leukemoid reaction, transient leukaemoid proliferation, transient abnormal myelopoiesis, acute myeloid leukemia (AML), acute megakaryoblastic leukemia (AMKL) (also known as erythro-megakaryoblastic leukaemia); common B-lineage acute lymphoblastic leukemia (ALL), polycythemia, thrombocythemia, myelodysplastic syndromes, myelofibrosis, hypereosinophilic syndrome (HES), chronic lymphocytic leukemia, prolymphocytic leukemia, hairy-cell leukemia, chronic myelogenous leukemia, other leukemias, and other myelogenous cancers.

Myelogenous cells retain the ability to enter and complete the cell cycle and proliferate. (Li B, Yang J, Andrews C, Chen Y X, Toofanfard P, Huang R W, Horvath E, Chopra H, Raza A, Preisler H D, Telomerase activity in preleukemia and acute myelogenous leukemia, Leuk Lymphoma February; 36(5-6):579-87 (2000); Clarkson B, Strife A, Cytokinetic considerations relevant to development of a successful therapeutic strategy in chronic myelogenous leukemia (CML), Leuk Lymphoma; 11 Suppl 1:101-7(1993)). Myeloproliferative diseases result when there is an upregulation of the cell cycle, resulting in abnormally increased proliferation of myelogenous cells. (Robbins S L, Cotran R S, Kumar V, Diseases of white cells, lymph nodes and spleen, in Pathologic Basis of Disease, ₃rd edition, edited by Robbins S L, Cotran R S, Kumar V, pp. 653-704, W.B. Saunders, Philadelphia, Pa. (1984)). According to the present invention, administration of one or more agents that prevent or inhibit an upregulation of the cell cycle, or abnormally increased proliferation of myleogenous cells, prevents or slows the progression or recurrence of myeloproliferative diseases.

In accordance with the present invention, and contrary to conventional teachings, increased levels, production, activity or function of LH and/or FSH contributes to myeloproliferative diseases by stimulating abnormally increased proliferation of myelogenous cells. For example, LH receptors are expressed in lymphocytes. (E.g., Lin J, et al., Lymphocytes from pregnant women express human chorionic gonadotropin/leutinizing hormone receptor gene, Mol Cell Endocrinol. Apr. 28, 1995; 111(1):R13-7). Also, LH and FSH have been shown to stimulate cell proliferation or differentiation in myelogenous cells. (Athreya B H, Rettig P, Williams W V, Hypophyseal-pituitary-adrenal axis in autoimmune and rheumatic diseases, Immunol Res; 18(2):93-102 (1998); Hotakainen P K, Serlachius E M, Lintula S I, Alfthan H V, Schroder J P, Stenman U E, Expression of luteinizing hormone and chorionic gonadotropin beta-subunit messenger-RNA and protein in human peripheral blood leukocytes, Mol Cell Endocrinol Apr. 25; 162(1-2):79-85 (2000)). In addition, at least one study suggests that an increase in the production or activity of LH and FSH stimulates cell proliferation in lymphocytes, a type of myelogenous cell. (Lin J, et al., Lymphocytes from pregnant women express human chorionic gonadotropin/leutinizing hormone receptor gene, Mol Cell Endocrinol. Apr. 28, 1995; 111(1):R13-7). Moreover, individuals with Down's syndrome, who have elevated levels of gonadotropins as compared to the general population, have a 10- to 20-fold increased risk of developing myelogenous neoplasms as compared to the general population. (Down syndrome and leukemia, Leukemia; 6 Suppl 1:5-7 (1992); Zipursky A, Poon A, Doyle J, Leukemia in Down syndrome: a review, Pediatr Hematol Oncol 9:139-49 (1992); Avet-Loiseau H, Mechinaud F, Harousseau J L. Clonal hematologic disorders in Down syndrome: a review, J Pediatr Hematol Oncol 17:19-24 (1995)).

Accordingly, the present invention encompasses preventing or treating a myeloproliferative disease, or preventing or slowing proliferation of myelogenous cells, by administering one or more LH/FSH-inhibiting agents, including those identified above, that decrease or regulate the blood level, production, function or activity or LH or FSH, or both.

Also in accordance with the present invention, and contrary to conventional teachings, an increased blood level, production, function or activity of activin or decreased levels, production, function, or activity of inhibin or follistatin, is associated with stimulating abnormally increased proliferation of myelogenous cells, which leads to myeloproliferative diseases. Accordingly, the present invention also encompasses preventing or treating a myeloproliferative disease, or preventing or slowing proliferation of myelogenous cells, by administering one or more activin-inhibiting agents, including those identified above, that decrease the blood level, production, function or activity of activin, or administering one or more inhibin-promoting agents or follistatin-promoting agents, including those identified above, that increase the levels, production, function or activity of inhibin or follistatin.

5. Osteoarthritis

The present invention also encompasses treating or preventing osteoarthritis, a disease associated with senescence. Osteoarthritis is a degenerative disease affecting virtually any joint in the body, characterized by inappropriate remodeling of joint tissue, including erosion of cartilage, formation of large calcified bone spurs, and the increased proliferation of cartilage cells, synovial intima cells (resulting in hyperplasia and hypertrophy), fibroblasts (resulting in increased production of collagen fibrils and fibrosis), and endothelial cells (resulting in blood vessel growth and hypervascularity) (Dijkgraaf L C, et al., Ultrastructural characteristics of the synovial membrane in osteoarthritic temporomandibular joints. Journal of Oral & Maxillofacial Surgery. 55(11):1269-79; discussion 1279-80 (1997); Kerin A, et al., Molecular basis of osteoarthritis: biomechanical aspects. Cell and Molecular Life Sciences 59(1):27-35 (2002); Hedbom E, Hauselmann H J, Molecular aspects of pathogenesis in osteoarthritis: the role of inflammation. Cell and Molecular Life Sciences. 59(1):45-53 (2002)). This joint tissue remodeling results in pain, deformity, and limitation of motion. (Silver F H, et al., Relationship among biomechanical, biochemical, and cellular changes associated with osteoarthritis. Critical Reviews in Biomedical Engineering. 29(4):373-91 (2001))

Research has focused on inhibiting the extensive joint tissue remodeling that occurs as part of osteoarthritis by administering sex steroid hormones, i.e. testosterone, progesterone and/or estrogen. For example, it is known that new bone formation and the closing of growth plates at the ends of long bones in post-pubertal adults requires the presence of the sex steroid hormones. (Smith E P, et al., Estrogen resistance caused by a mutation in the estrogen-receptor gene in a man, New England Journal of Medicine, 331:1056-1061 (1994); Somjen D, et al., Age dependence and modulation by gonadectomy of the sex-specific response of rat diaphyseal bone to gonadal steroids, Endocrinology 134(2):809-14 (1994)) There is also evidence that estrogen may maintain the continuity of joint cartilage (Turner A S, et al., Biochemical effects of estrogen on articular cartilage in ovariectomized sheep, Osteoarthritis and Cartilage 5:63-69 (1997)). In addition, it is believed that sex steroid hormones play a role in bone, cartilage, and joint tissue growth and structure. (Corvol M, et al., Bone and cartilage responsiveness to sex steroid hormones, Journal of Steroid Biochemistry and Molecular Biology. 43(5):415-8 (1992)).

Recent studies also have provided evidence that estrogen plays a role in regulating bone resorption and that testosterone and estrogen maintain bone formation. (Sypniewska G, et al., Bone turnover markers and estradiol level in postmenopausal women, Clinical Chemistry Laboratory Medicine, 38(11):1115-1119 (2000); D'Amore M, et al., Sex hormones and male osteoporosis: a physiologic prospective for prevention and therapy, Minerva Medicine, 91(11-12):283-289 (2000)). In one study, testosterone and estrogen production was blocked in a group, of 59 elderly men, who were then administered physiological doses of one or both of these steroids. The results showed that estrogen levels in males correlate directly with bone mineral density, which in turn correlates with susceptibility to osteoarthritis. (Khola S, Melton L J, Riggs B L, Estrogens and bone health in men, Calcif. Tissue Int. 69:189-192 (2001)). Other studies have shown a similar correlation between levels of sex steroids and osteoarthritis. (Sowers M F, et al., Association of bone mineral density and sex hormone levels with osteoarthritis of the hand and knee in premenopausal women, American Journal of Epidemiology, 143(1):38-47 (1996); Spector T D, et al., Endogenous sex steroid levels in women with generalised osteoarthritis, Clinical Rheumatology, 10(3):316-9 (1991)).

Nonetheless, the results of sex steroid administration in treating osteoarthritis have been, at best, mixed. While a few studies suggest that estrogen replacement is beneficial in the treatment of osteoarthritis (e.g., Wluka A E, et al., Users of estrogen replacement therapy have more knee cartilage than non-users, Annals of Rheumatoid Disease, 60(4):332-6 (2001); Felson D T, Nevitt M C, The effects of estrogen on osteoarthritis, Current Opinions in Rheumatology, 10(3):269-72 (1998)), a number of other studies suggest that estrogen replacement has no benefit (e.g., Nevitt M C, et al., The effect of estrogen plus progestin on knee symptoms and related disability in postmenopausal women: The Heart and Estrogen/Progestin Replacement Study, a randomized, double-blind, placebo-controlled trial, Arthritis and Rheumatology, 44(4):811-8 (2001); Maheu E, et al., Hand osteoarthritis patients characteristics according to the existence of a hormone replacement therapy, Osteoarthritis and Cartilage, 8 Suppl A:S33-7 (2000); Erb A, et al., Hormone replacement therapy and patterns of osteoarthritis: baseline data from the Ulm Osteoarthritis Study, Annals of Rheumatoid Disease, 59(2):105-9 (2000)). Moreover, estrogen replacement therapy has been associated with an increased risk of breast cancer. (Chen C L, et al., Hormone replacement therapy in relation to breast cancer, Journal of the American Medical Association, 287(6):734-41 (2002)).

In accordance with the present invention, and contrary to conventional teachings, an upregulation in the cell cycle, caused by increased mitogenic stimulus, contributes to osteoarthritis by causing increased inappropriate remodeling of joint tissue and increased proliferation of cartilage cells, synovial intima cells, fibroblasts, and endothelial cells.

Also in accordance with the present invention, and contrary to conventional teachings, upregulation of the cell cycle associated with extensive remodeling of joint tissue, characteristic of osteoarthritis, results from an increase in the blood level, production, activity or function of LH and/or FSH. For example, one study has shown, a three- to four-fold increase in LH serum concentrations and a four- to eighteen-fold increase in FSH serum concentrations in elderly women. (Chakravarti S, Collins W P, Forecast J D, Newton J R, Oram D H, Studd J W, Hormonal profiles after the menopause, Br Med J Oct. 2, 1976; 2(6039):784-7) Likewise, elderly men also experience a greater than two-fold, and three-fold, increase in LH and FSH serum concentrations, respectively. (Neaves et al. 1984). In addition, mRNA levels of leutinizing hormone releasing hormone (LHRH) in the hypothalamus of elderly women are increased. (Rance N E, Uswandi S V, Gonadotropin-releasing hormone gene expression is increased in the medial basal hypothalamus of postmenopausal women, Journal of Clinical Endocrinology and Metabolism, 81(10):3540-6 (1996)). Also, a study has shown that LH stimulates the growth of chondrocytes (cartilage cells) in rabbit epiphyseal growth plates. (Webber R J, Sokoloff L, In vitro culture of rabbit growth plate chondrocytes: age-dependence of response to fibroblast growth factor and “chondrocyte growth factor,” Growth 45:252-268 (1981)). According to the present invention, an increase in blood level, production, function or activity of LH or FSH increases the rate of joint tissue growth, thereby increasing synovial inflammation, inappropriate tissue formation at joints and the occurrence and severity of joint tissue remodeling characteristic of osteoarthritis.

Accordingly, the present invention encompasses a method of treating or preventing osteoarthritis, or preventing or slowing proliferation of cartilage cells, synovial intima cells, fibroblasts, or endothelial cells, by administering one or more LH/FSH-inhibiting agents, including those identified above, that decrease or regulate the blood level, production, function or activity or LH or FSH, or both.

Also in accordance with the present invention, and contrary to conventional teachings, increases in the blood level, production, function or activity of activin, or decreases in the blood level, production, function, or activity of inhibin or follistatin, are associated with upregulation of the cell cycle and stimulating increased inappropriate remodeling of bone that is characteristic of osteoarthritis. Activin binds, for example, to bone morphogenic protein (BMP) receptors, which are present on cells associated with bone remodeling. In addition, secretion of high levels of activin during gestation has been shown to increase cell proliferation in several tissues. (Qu J, Thomas K, Inhibin and activin production in human placenta, Endocrine Reviews 16:485-507 (1995)). During the adult reproductive period, the function of activin is counteracted by inhibin or follistatin. (Halvorson, L M & Chin W W, Gonadotropic hormones: biosynthesis, secretion, receptors, and action, in Reproductive Endocrinology, 4th ed. Yen S S C, Jaffe R B & Barbieri L L, eds.: 94-97, W.B. Saunders, Philadelphia, Pa. (1999)).

Accordingly, the present invention also encompasses a method of treating or preventing osteoarthritis, or preventing or slowing proliferation of cartilage cells, synovial intima cells, fibroblasts, or endothelial cells, by administering one or more activin-inhibiting agents, including those identified above, that decrease the blood level, production, function or activity of activin, or administering one or more inhibin-promoting agents or follistatin-promoting agents, including those identified above, that increase the levels, production, function or activity of inhibin or follistatin.

6. Osteoporosis

The present invention further encompasses a method for treating or preventing osteoporosis, a disease associated with senescence. Osteoporosis is a major public health concern for Americans, affecting approximately 44 million people, about 68% of whom are female. (Brunader R, Shelton D K, Radiologic bone assessment in the evaluation of osteoporosis, Am Fam Physician 65:1357-64 (2002)). This disease is responsible for more than 1.5 million bone fractures annually. (NORA study sounds alarm on risk of osteoporotic fractures, Dis Manag Advis 8:17-21 (2002)).

Osteoporosis (derived from the Latin meaning “porous bone”) is characterized by loss of bone mass and structural deterioration and weakness of bone tissue, leading to increased susceptibility to bone fractures, particularly in the bones of the hip, spine and wrist. (Sherman S, Preventing and treating osteoporosis: strategies at the millennium, Ann N Y Acad Sci, December; 949: 188-97 (2001)). Bone tissue is remodeled continuously throughout life in order to maintain its anatomical and structural integrity. (Manolagas S C, Jilka R L, Bone marrow, cytokines, and bone remodeling, New Eng J Med 332:305-311 (1995)). Old bone tissue is resorbed by the action of cells known as osteoclasts while new bone tissue is formed by the action of cells known as osteoblasts. Under normal conditions, bone tissue remodeling is cyclical, with osteoclasts removing bone tissue by acidification and proteolytic digestion, and osteoblasts secreting osteoid (a matrix of collagen and other proteins), which is eventually mineralized to form new bone tissue.

During childhood and puberty in humans, new bone is added faster than old bone is resorbed, causing bones to become larger, more massive, and more dense. (Saggese G, Baroncelli G I, Bertelloni S, Puberty and bone development, Best Pract Res Clin Endocrinol Metab 16:53-64 (2002)). During the adult reproductive period, the rate of resorption of old bone and the rate of addition of new bone are approximately the same, keeping the size, mass, and density of bones relatively constant. (Raisz L G, Kream B E, Lorenzo J A, Metabolic Bone Disease, in Williams Textbook of Endocrinology, p. 1211-1239, edited by Wilson J D, Foster D W, Kronenberg H M, Larsen P R, WB Saunders Co., Philadelphia, Pa. (1998)). During this period, approximately 25 percent of trabecular bone and approximately 3 percent of cortical bone is resorbed and replaced every year. (Manolagas S C, Jilka R L, Bone marrow, cytokines, and bone remodeling, New Eng J Med 332:305-311 (1995)).

Beginning in approximately the fourth or fifth decade of life, the rate of bone resorption begins to exceed the rate of new bone formation, leading to bone loss and structural deterioration and weakness of bone tissue characteristic of osteoporosis. (Raisz L G, Kream B E, Lorenzo J A, Metabolic bone disease, in Williams Textbook of Endocrinology, p. 1211-1239, 1998, edited by Wilson J D, Foster D W, Kronenberg H M, Larsen P R, WB Saunders Co., Philadelphia, Pa.). Bone loss tends to be more rapid in women, especially during the first few years after menopause. (Cooper C, Melton L J, Epidemiology of osteoporosis, Trends Endocrinol Metab 3:224-228 (1992)). However, bone loss occurs in both sexes with advancing age. (Melton L J, Chrischilles E A, Cooper C, Lane A W, Riggs B L, Perspective: How many women have osteoporosis?, J Bone Miner Res 7:1005-10 (1992)). Risk factors for osteoporosis include, but are not limited to, gender, age, body size (with small, thin-boned women at greater risk), ethnicity (with Caucasians and Asians at greater risk), family history, low estrogen or testosterone levels, anorexia, low calcium and vitamin D diets, cigarette smoking and excessive alcohol use. (Raisz L G, Kream B E, Lorenzo J A, Metabolic bone disease, p. 1221-1222; Messinger-Rapport B J, Thacker H L, Prevention for the older woman: A practical guide to prevention and treatment of osteoporosis, Geriatrics 57:16-8, 21-4 (2002); Zipfel S et al., Herzog W, Osteoporosis in eating disorders: a follow-up study of patients with anorexia and bulimia nervosa, J Clin Endocrinol Metab 86:5227-33 (20Q1); Brown A F et al., Ethnic differences in hormone replacement prescribing patterns, J Gen Intern Med 14:663-9 (1999); Moniz C, Alcohol and bone, Br Med Bull 50:67-75 (1994); Ward K D, Klesges R C, A meta-analysis of the effects of cigarette smoking on bone mineral density, Calcif Tissue Int 68:259-70 (2001)).

Current treatments for osteoporosis include, but are not limited to, the administration of estrogen or other sex steroid replacements, bisphosphonates (such as alendronate sodium and risedronate sodium), selective estrogen receptor modulators (such as raloxifene), calcitonin, calcium, and vitamin D. (Lafferty F W, Fiske M E, Postmenopausal estrogen replacement: a long-term cohort study, Am J Med, 97:66-77 (1994); Chestnut C H 3rd et al., Alendronate treatment of the postmenopausal osteoporotic woman: effect of multiple dosages on bone mass and bone remodeling, Am J Med 99:144-52 (1995); Maricic M, Gluck O, Review of raloxifene and its clinical applications in osteoporosis, Expert Opin Pharmacother 3:767-75 (2002); Civitelli R et al., Bone turnover in postmenopausal osteoporosis: Effect of calcitonin treatment, J Clin Invest. 82:1268-74 (1988); Prentice A, What are the Dietary Requirements for Calcium and Vitamin D?, Calcif Tissue Int 70:83-8 (2002)).

In accordance with the present invention, and contrary to conventional teachings, an upregulation of the cell cycle contributes to osteoporosis by causing increased proliferation of osteoclasts (which resorb bone) and/or decreased proliferation of osteoblasts (which create bone), likely due to increased expression of gonadotropin receptors on osteoclasts as compared with osteoblasts. In accordance with the present invention, and contrary to conventional teachings, an age-related increase in the blood level, production, function, or activity of LH or FSH contributes to osteoporosis by causing increased proliferation of osteoclasts and/or decreased proliferation of osteoblasts, which leads to bone resorption. For example, one study has shown a three- to four-fold increase in LH serum concentrations and a four- to eighteen-fold increase in FSH serum concentrations in elderly women. (Chakravarti S, Collins W I P, Forecast J D, Newton J R, Oram D H, Studd J W, Hormonal profiles after the menopause, Br Med J, Oct. 2, 1976; 2(6039):784-7). Likewise, elderly men experience a greater than two-fold, and three-fold, increase in LH and FSH serum concentrations, respectively. (Neaves et al. 1984). In addition, mRNA levels of leutinizing hormone releasing hormone (LHRH) in the hypothalamus of elderly women are increased. (Rance N E, Uswandi S V, Gonadotropin-releasing hormone gene expression is increased in the medial basal hypothalamus of postmenopausal women, Journal of Clinical Endocrinology and Metabolism, 81(10):3540-6 (1996)).

Accordingly, the present invention encompasses a method of preventing or treating osteoporosis, or preventing or slowing proliferation of osteoclasts, or increasing or promoting proliferation of osteoblasts, by administering one or more LH/FSH-inhibiting agents, including those identified above, that decrease or regulate the blood level, production, function or activity or LH or FSH, or both.

Also in accordance with the present invention, and contrary to conventional teachings, increased blood level, production, function or activity of activin or decreased blood level, production, function, or activity of inhibin or follistatin are associated with increased proliferation of osteoclasts and/or decreased proliferation of osteoblasts, which leads to bone resorption. Activin binds, for example, to bone morphogenic protein (BMP) receptors, which are present on cells associated with bone remodeling. In addition, secretion of high levels of activin during gestation has been shown to increase cell proliferation in several tissues. (Qu J, Thomas K, Inhibin and activin production in human placenta, Endocrine Reviews 16:485-507 (1995)). During the adult reproductive period, the function of activin is counteracted by inhibin or follistatin. (Halvorson, L M & Chin W W, Gonadotropic hormones: biosynthesis, secretion, receptors, and action, in Reproductive Endocrinology, 4th ed. Yen S S C, Jaffe R B & Barbieri R L, eds.: 94-97. W.B. Saunders, Philadelphia, Pa. (1999)).

Accordingly, the present invention encompasses a method of preventing or treating osteoporosis, or preventing or slowing proliferation of osteoclasts, or increasing or promoting proliferation of osteoblasts, by administering one or more activin-inhibiting agents, including those identified above, that decrease the blood level, production, function or activity of activin, or administering one or more inhibin-promoting agents or follistatin-promoting agents, including those identified above, that increase the levels, production, function or activity of inhibin or follistatin.

The present invention further encompasses a method of preventing or treating osteoporosis, or preventing or slowing proliferation of osteoclasts, or increasing or promoting proliferation of osteoblasts, by administering one of the aforementioned cell cycle inhibitors that prevent or inhibit cells from entering into the cell cycle. Such agents include, but are not limited to, low density lipoprotein receptor related protein receptor associated protein (“RAP”); a vaccine or antibody against proteins involved in promoting cell division (e.g. against cell cycle proteins such as CDK); taxol; vitamin A; hydroxyurea; colchicines; cholesterol lowering drugs, such as lovastatin or provastatin; and analogs, metabolites, precursors, and salts thereof.

7. Brain Damage Associated With Acute Brain Injury

The present invention also encompasses treating or preventing brain damage associated with acute brain injury, including both those brain injuries associated with senescence and those brain injuries not associated with senescence. In this specification, by “acute brain injury” is meant any damage to the brain that occurs suddenly or over a short period of time. Examples of such injury include, but are not limited to, the damage that results from stroke, hypoxia, choking, head trauma, concussion, or any loss of consciousness.

Acute brain injury stimulates the brain's repair mechanisms, one of which is upregulation of the cell cycle. (Chirumamilla S, Sun D, Bullock M R, Colello R J, Traumatic brain injury induced cell proliferation in the adult mammalian central nervous system, J Neurotrauma June. 2002; 19(6):693-703; Kernie S G, Erwin T M, Parada L F, Brain remodeling due to neuronal and astrocytic proliferation after controlled cortical injury in mice, J Neurosci Res 2001 Nov 1; 66(3):317-26). This occurs both with brain injuries associated with senescence (such as a stroke) and with brain injuries not associated with senescence (such as head trauma). Cyclin-dependent kinases (“CDKs”) are also present and are commonly known to regulate cell cycling. (Kaya S S, Mahmood A, Li Y, Yavuz E, Chopp M, Expression of cell cycle proteins (cyclin D1 and cdk4) after controlled cortical impact in rat brain, J Neurotrauma December 1999; 16(12):1187-96; Koguchi K, Nakatsuji Y, Nakayama K, Sakoda S, Modulation of astrocyte proliferation by cyclin-dependent kinase inhibitor p 27(Kip1), Glia February 2002; 37(2):93-104). While in some tissues, such as the intestinal mucosa, cell division is necessary for normal function, upregulation of the cell cycle in the brain, where the majority of cells are terminally differentiated, could be detrimental, especially after an acute brain injury. Although terminally differentiated neuronal cells may be able to enter the cell cycle, they are unable to complete the process, leaving the cells in a compromised position and causing diminished cellular function or apoptosis (i.e., cell death). (Multani A S, Ozen M, Narayan S, Kumar V, Chandra J, McConkey D J, Newman R A, Pathak S, Caspase-dependent apoptosis induced by telomere cleavage and TRF2 loss, Neoplasia July-August; 2(4):339-45 (2000)).

In accordance with the present invention, and contrary to conventional teachings, upregulation of the cell cycle in neuronal cells is caused, at least in part, by an increase in blood levels, production, function or activity of LH or FSH. For example, as discussed above, a study was conducted to confirm that the presence of LH in neuroblastoma cells (i.e., neuronal tumor cells) stimulates cell proliferation. In that study, various amounts of LH, ranging from 0 to 160 mIU, were added to samples of neuroblastoma cells cultured in serum free media. The cultures were BrdU labeled to indicate the amount of cell division. As shown in FIG. 2, those cultures that received non-zero amounts of LH had a significantly increased rates of cell division as compared with cells that received no LH, with the highest rates occurring at LH concentrations of 5-40 mIU. Cells that received physiological concentrations of LH (5-10 mIU/ml) had a rate of cell proliferation approximately 50% greater than cells that received no LH.

Also, as discussed above, a second study was conducted to confirm that the administration to neuroblastoma cells of leuprolide, a GnRH analog that decreases the level, production, function or activity of LH and FSH, decreases proliferation of those cells. FIG. 3 illustrates the results for neuroblastoma cells exposed, in vitro, to leuprolide at a concentration of about 10 nM, which is approximately equivalent to a therapeutically effective blood level of leuprolide, according to the present invention. As shown in FIG. 3, after three days, neuroblastoma cells that received leuprolide had almost three-times less cell proliferation than neuroblastoma cells that received no leuprolide.

Accordingly, the present invention encompasses preventing or treating brain damage associated with acute brain injury by administering one or more LH/FSH-inhibiting agents, including those identified above, that decrease or regulate the blood level, production, function or activity or LH or FSH, or both.

Also in accordance with the present invention, and contrary to conventional teachings, increased blood level, production, function or activity of activin, or decreased levels, production, function, or activity of inhibin or follistatin is associated with stimulating neuronal cells to enter the cell cycle. For example, secretion of high levels of activin during gestation has been shown to increase cell cycling in several tissues. (Qu J, Thomas K, Inhibin and activin production in human placenta, Endocrine Reviews 16:485-507 (1995)). During the adult reproductive period, the function of activin is counteracted by inhibin and/or follistatin. (Halvorson, L M & Chin W W, Gonadotropic hormones: biosynthesis, secretion, receptors, and action, in Reproductive Endocrinology, 4th ed. Yen S S C, Jaffe R B & Barbieri R L, eds.: 94-97. W.B. Saunders, Philadelphia, Pa. (1999)).

Accordingly, the present invention also encompasses preventing or treating brain damage associated with acute brain injury by administering one or more activin-inhibiting agents, including those identified above, that decrease the blood level, production, function or activity of activin, or administering one or more inhibin-promoting agents or follistatin-promoting agents, including those identified above, that increase the levels, production, function or activity of inhibin or follistatin.

The present invention further encompasses a method of treating or preventing brain damage associated with acute brain injury by administering one of the aforementioned cell cycle inhibitors that prevent or inhibit cells from entering into the cell cycle. Such agents include, but are not limited to, low density lipoprotein receptor related protein receptor associated protein (“RAP”); a vaccine or antibody against proteins involved in promoting cell division (e.g. against cell cycle proteins such as CDK); taxol; vitamin A; hydroxyurea; colchicines; cholesterol lowering drugs, such as lovastatin or provastatin; and analogs, metabolites, precursors, and salts thereof.

In the treatment of each of the foregoing diseases associated with senescence, as well as any other diseases associated with senescence, the LH/FSH-inhibiting agents, activin-inhibiting agents, inhibin-promoting agents, and follistatin-promoting agents are administered in therapeutically effective combinations, quantities and dosage regimens that achieve a blood level, production, function or activity of LH, FSH, activin, inhibin, and/or follistatin at or near the target blood level, target production, target function or target activity of LH, FSH, activin, inhibin, and/or follistatin, as discussed above. These agents also may be co-administered with one or more sex steroids, as described above.

While various embodiments of the present invention have been described above, it should be understood that they have been presented by way of example only, and not by limitation. For example, the present invention is not limited to the agents or diseases illustrated or described. As such, the breadth and scope of the present invention should not be limited to any of the above-described exemplary embodiments, but should be defined in accordance with the following claims and their equivalents.

What is claimed is:

1. A method of treating a disease associated with senescence in a human subject, comprising:
administering to the subject a therapeutically effective amount of an agent that decreases the subject's blood level of FSH and that decreases the subject's blood level of LH; wherein:
the agent is administered to the subject such that a therapeutically effective blood level of the agent is achieved;
the agent comprising one or more of GnRH, leuprolide, triptorelin, buserelin, nafarelin, desorelin, histrelin, or goserelin, or a physiologically acceptable analog, metabolite, precursor or salt thereof, and
the disease selected from the group consisting of: polyps of the colon, colorectal cancer, myeloproliferative diseases, Hodgkin's disease, multiple myeloma, transient myeloproliferative disorder (TMD; also known as transient myeloproliferative syndrome), congenital transient leukemia, congenital leukemoid reaction, transient leukaemoid proliferation, transient abnormal myelopoiesis, acute myeloid leukemia (AML), acute megakaryoblastic leukemia (AMKL; also known as erythro-megakaryoblastic leukaemia), polycythemia, thrombocythemia, myelodysplastic syndromes, myelofibrosis, hypereosinophilic syndrome (HES), hairy-cell leukemia, chronic myelogenous leukemia, chronic lymphocytic leukemia, prolymphocytic leukemia, common B lineage acute lymphoblastic leukemia, other leukemias, other myelogenous cancers, osteoarthritis, cataracts, macular degeneration, hearing loss, stroke, peripheral neuropathy, COPD, hypertension, type 2 diabetes, sarcopenia, hypertension, primary pulmonary hypertension, congestive heart failure, left ventricular hypertrophy, cardiac valvular disease, esophagitis, esophageal stricture, gastroparesis, chronic pancreatitis, hypercholesterolemia, hypertriglyceridemia, cirrhosis of the liver, hepatitis, cholelithiasis, cholecystitis, ulcerative colitis, inflammatory bowel disease, Crohn's disease, fibromyalgia, obesity, renal failure, proteinuria, gout, hyperuricemia, membranous nephropathy, polyarteritis nodosa, polymyalgia rheumatica, rheumatoid arthritis, progressive systemic sclerosis, migraine headaches, male pattern baldness, sarcoidosis, Wegener granulomatosis, amyloidosis, dermatomyositis, graft versus host disease, systemic lupus erythematosus, seborrheic dermatitis, psoriasiform eczematous dermatitis, papulosquamous eczematous dermatitis, psoriasis, seborrheic keratosis, anagen effluvium, dysphagia, Barrett esophagus, achalasia, Chagas disease, myasthenia gravis, multiple sclerosis, transverse myelitis, Parkinson's disease, squamous cell carcinoma of the lung, adenocarcinoma of the lung, large cell carcinoma of the lung, small cell carcinoma of the lung, esophageal cancer, gastric cancer, pancreatic cancer, hepatocellular cancer, gallbladder carcinomas, non-Hodgkin's lymphoma, follicular lymphoma, small lymphocytic lymphoma, mantle cell lymphoma, marginal zone lymphoma, diffuse large cell lymphoma, multiple myeloma, Ewing's sarcoma, chondrosarcomas, osteosarcomas, renal cell carcinoma, bladder carcinoma, squamous cell carcinoma of the head and neck, salivary gland tumors, Sjogren syndrome, mixed connective tissue disease, Whipple disease, Paget's disease of bone, osteomalaci, decreased immune function and decreased muscle mass.

2. The method of claim 1, wherein the administering of the therapeutically effective amount of the agent is effective to decrease the blood level of LH or FSH in the subject to less than approximately 0.7 mIU/mL.

3. The method of claim 1, wherein the subject is male.

4. The method of claim 3, wherein the administering of the therapeutically effective amount of the agent is effective to decrease the blood level of LH in the subject to approximately 10 mIU/mL.

5. The method of claim 3, wherein the administering of the therapeutically effective amount of the agent is effective to decrease the blood level of LH or FSH in the subject to less than approximately 0.7 mIU/mL.

6. The method of claim 3, wherein the achieved therapeutically effective blood level of the administered agent is approximately 10 nM.

7. The method of claim 3, wherein the administering of the therapeutically effective amount of the agent is effective to decrease the blood level of FSH in the subject to approximately 2.0-22.6 mIU/mL.

8. The method of claim 3, wherein the administering of the therapeutically effective amount of the agent is effective to decrease the blood level of FSH in the subject to approximately 22.6 mIU/mL.

9. The method of claim 3, wherein the administering of the therapeutically effective amount of the agent is effective to decrease the blood level of LH in the subject to approximately 0-10 mIU/mL.

10. The method of claim 1, wherein the subject is female.

11. The method of claim 10, wherein the administering of the therapeutically effective amount of the agent is effective to decrease the blood level of LH in the subject to at or near 0.4 mIU/mL in the subject.

12. The method of claim 10, wherein the administering of the therapeutically effective amount of the agent is effective to decrease the blood level of LH or FSH in the subject to less than approximately 0.7 mIU/mL.

13. The method of claim 10, wherein the achieved therapeutically effective blood level of the administered agent is approximately 10 nM.

14. The method of claim 10, wherein the administering of the therapeutically effective amount of the agent is effective to decrease the blood level of FSH in the subject to approximately 2.9 mIU/mL.

15. The method of claim 10, wherein the administering of the therapeutically effective amount of the agent is effective to decrease the blood level of FSH in the subject to approximately 2.9-29.5 mIU/mL.

16. The method of claim 1 wherein the blood level of LH or FSH is decreased using a time-released dose of the agent such that a therapeutically effective blood level of the agent is achieved.

17. The method of claim 16, wherein the administering of the therapeutically effective amount of the agent is effective to decrease the blood level of LH in the subject to approximately 0-10 mIU/mL, and wherein the subject is male.

18. The method of claim 16, wherein the achieved therapeutically effective blood level of the administered agent is approximately 10 nM.

19. A method of treating a disease associated with senescence in a human subject, comprising:
administering to the subject a therapeutically effective amount of an agent that decreases the subject's blood level of LH in the subject to at or near 0 mIU/mL in order to treat a disease associated with senescence, the subject being a male over the age of 35 years old,
said agent comprising one or more of the following, or a physiologically acceptable analog, metabolite, precursor or salt thereof: GnRH; leuprolide; triptorelin; buserelin; nafarelin; desorelin; histrelin; or goserelin;
and wherein said disease is selected from the group consisting of: polyps of the colon, colorectal cancer, myeloproliferative diseases, Hodgkin's disease, multiple myeloma, transient myeloproliferative disorder (TMD; also known as transient myeloproliferative syndrome), congenital transient leukemia, congenital leukemoid reaction, transient leukaemoid proliferation, transient abnormal myelopoiesis, acute myeloid leukemia (AML), acute megakaryoblastic leukemia (AMKL; also known as erythro-megakaryoblastic leukaemia), polycythemia, thrombocythemia, myelodysplastic syndromes, myelofibrosis, hypereosinophilic syndrome (HES), hairy-cell leukemia, chronic myelogenous leukemia, chronic lymphocytic leukemia, prolymphocytic leukemia, common B lineage acute lymphoblastic leukemia, other leukemias, other myelogenous cancers, osteoarthritis, cataracts, macular degeneration, hearing loss, stroke, peripheral neuropathy, COPD, hypertension, type 2 diabetes, sarcopenia, hypertension, primary pulmonary hypertension, congestive heart failure, left ventricular hypertrophy, cardiac valvular disease, esophagitis, esophageal stricture, gastroparesis, chronic pancreatitis, hypercholesterolemia, hypertriglyceridemia, cirrhosis of the liver, hepatitis, cholelithiasis, cholecystitis, ulcerative colitis, inflammatory bowel disease, Crohn's disease, fibromyalgia, obesity, renal failure, proteinuria, gout, hyperuricemia, membranous nephropathy, polyarteritis nodosa, polymyalgia rheumatica, rheumatoid arthritis, progressive systemic sclerosis, migraine headaches, male pattern baldness, sarcoidosis, Wegener granulomatosis, amyloidosis, dermatomyositis, graft versus host disease, systemic lupus erythematosus, seborrheic dermatitis, psoriasiform eczematous dermatitis, papulosquamous eczematous dermatitis, psoriasis, seborrheic keratosis, anagen effluvium, dysphagia, Barrett esophagus, achalasia, Chagas disease, myasthenia gravis, multiple sclerosis, transverse myelitis, Parkinson's disease, squamous cell carcinoma of the lung, adenocarcinoma of the lung, large cell carcinoma of the lung, small cell carcinoma of the lung, esophageal cancer, gastric cancer, pancreatic cancer, hepatocellular cancer, gallbladder carcinomas, non-Hodgkin's lymphoma, follicular lymphoma, small lymphocytic lymphoma, mantle cell lymphoma, marginal zone lymphoma, diffuse large cell lymphoma, multiple myeloma, Ewing's sarcoma, chondrosarcomas, osteosarcomas, renal cell carcinoma, bladder carcinoma, testicular carcinoma, seminoma, nonseminoma, squamous cell carcinoma of the head and neck, salivary gland tumors, Sjogren syndrome, mixed connective tissue disease, Whipple disease, Paget's disease of bone, osteomalaci, decreased immune function and decreased muscle mass.

20. A method of treating a disease associated with senescence in a human subject, comprising:
administering to the subject a therapeutically effective amount of an agent that decreases the subject's blood level of FSH and that decreases the subject's blood level of LH to at or near 0.4 mIU/mL in order to treat a disease associated with senescence,
the subject being a female over the age of 35 years old,
the agent comprising one or more of GnRH, leuprolide, triptorelin, buserelin, nafarelin, desorelin, histrelin, or goserelin, or a physiologically acceptable analog, metabolite, precursor or salt thereof,
the disease selected from the group consisting of: polyps of the colon, colorectal cancer, myeloproliferative diseases, Hodgkin's disease, multiple myeloma, transient myeloproliferative disorder (TMD; also known as transient myeloproliferative syndrome), congenital transient leukemia, congenital leukemoid reaction, transient leukaemoid proliferation, transient abnormal myelopoiesis, acute myeloid leukemia (AML), acute megakaryoblastic leukemia (AMKL; also known as erythro-megakaryoblastic leukaemia), polycythemia, thrombocythemia, myelodysplastic syndromes, myelofibrosis, hypereosinophilic syndrome (HES), hairy-cell leukemia, chronic myelogenous leukemia, chronic lymphocytic leukemia, prolymphocytic leukemia, common B lineage acute lymphoblastic leukemia, other leukemias, other myelogenous cancers, osteoarthritis, cataracts, macular degeneration, hearing loss, stroke, peripheral neuropathy, COPD, hypertension, type 2 diabetes, sarcopenia, hypertension, primary pulmonary hypertension, congestive heart failure, left ventricular hypertrophy, cardiac valvular disease, esophagitis, esophageal stricture, gastroparesis, chronic pancreatitis, hypercholesterolemia, hypertriglyceridemia, cirrhosis of the liver, hepatitis, cholelithiasis, cholecystitis, ulcerative colitis, inflammatory bowel disease, Crohn's disease, fibromyalgia, obesity, renal failure, proteinuria, gout, hyperuricemia, membranous nephropathy, polyarteritis nodosa, polymyalgia rheumatica, rheumatoid arthritis, progressive systemic sclerosis, migraine headaches, male pattern baldness, sarcoidosis, Wegener granulomatosis, amyloidosis, dermatomyositis, graft versus host disease, systemic lupus erythematosus, seborrheic dermatitis, psoriasiform eczematous dermatitis, papulosquamous eczematous dermatitis, psoriasis, seborrheic keratosis, anagen effluvium, dysphagia, Barrett esophagus, achalasia, Chagas disease, myasthenia gravis, multiple sclerosis, transverse myelitis, Parkinson's disease, squamous cell carcinoma of the lung, adenocarcinoma of the lung, large cell carcinoma of the lung, small cell carcinoma of the lung, esophageal cancer, gastric cancer, pancreatic cancer, hepatocellular cancer, gallbladder carcinomas, non-Hodgkin's lymphoma, follicular lymphoma, small lymphocytic lymphoma, mantle cell lymphoma, marginal zone lymphoma, diffuse large cell lymphoma, multiple myeloma, Ewing's sarcoma, chondrosarcomas, osteosarcomas, renal cell carcinoma, bladder carcinoma, squamous cell carcinoma of the head and neck, salivary gland tumors, Sjogren syndrome, mixed connective tissue disease, Whipple disease, Paget's disease of bone, osteomalaci, decreased immune function and decreased muscle mass.
